(19) United States
(12) Patent Application Publication
(10) Pub. No.: US 2026/0305916 A1
(43) Pub. Date: Oct. 8, 2026

(54) IMAGE PROCESSING ADAPTER FOR ENDOSCOPE

(71) Applicant: GYRUS ACMI, INC D/B/A OLYMPUS SURGICAL TECHNOLOGIES AMERICA, Westborough, MA (US)

(72) Inventors: Robert E. Ailinger, Norwood, MA (US); Eric A. Frattura, Shrewsbury, MA (US); Jeffery D. Penman, Ogden, UT (US); John P. Whidden, Salt Lake City, UT (US); Caleb S. Roundy, Riverton, UT (US)

(21) Appl. No.: 19/481,324

(22) PCT Filed: May 2, 2024
(86) PCT No.: PCT/US2024/027461

§ 371 Date: Nov. 4, 2025

Related U.S. Application Data
(60) Provisional application No. 63/500,046, filed on May 4, 2023.
(60) Provisional application No. 63/590,638, filed on Oct. 16, 2023.

(51) Int. Cl. A61B 1/00

(52) U.S. Cl. CPC A61B 1/00124; A61B 1/000095; A61B 1/00059; A61B 1/00126; A61B 1/00131

(57) ABSTRACT

An adapter for an endoscope system comprises a housing, an input device for receiving imaging signal inputs from an endoscope, an imaging signal processor configured to receive imaging signal inputs and perform an image processing technique to imaging signal inputs to generate imaging signal outputs, the imaging signal processing technique configured to alter a display characteristic of imaging signal inputs, and an output coupler for providing imaging signal outputs to an imaging and control system. A method for transforming a flat image to a distorted image comprises mapping pixels of the flat image to a first table correlating the pixels to locations in the flat image, correlating the locations of the first table to locations in a second table for the distorted image, and generating the distorted image by moving pixels from the locations of the first table to associated locations in the second table.

### PRIORITY CLAIM

This application claims the benefit of priority to U.S. Provisional Patent Application Ser. No. 63/500,046, filed May 4, 2023, and U.S. Provisional Patent Application Ser. No. 63/590,638, filed Oct. 16, 2023, the contents of which are incorporated herein by reference.

### TECHNICAL FIELD

The present disclosure relates generally to medical devices comprising elongate bodies configured to be inserted into incisions or openings in anatomy of a patient to provide diagnostic or treatment operations.

More specifically, the present disclosure relates to systems and devices for establishing connectivity between medical devices and imaging and control systems.

### BACKGROUND

Endoscopes can be used for one or more of 1) providing passage of other devices, e.g., therapeutic devices or tissue collection devices, toward various anatomical portions, and 2) imaging of such anatomical portions. Such anatomical portions can include the gastrointestinal tract (e.g., esophagus, stomach, duodenum, pancreaticobiliary duct, intestines, colon, and the like), renal area (e.g., kidney(s), ureter, bladder, urethra) and other internal organs (e.g., reproductive systems, sinus cavities, submucosal regions, respiratory tract), and the like.

Conventional endoscopes can be involved in a variety of clinical procedures, including, for example, illuminating, imaging, detecting and diagnosing one or more disease states, providing fluid delivery (e.g., saline or other preparations via a fluid channel) toward an anatomical region, providing passage (e.g., via a working channel) of one or more therapeutic devices for sampling or treating an anatomical region, and providing suction passageways for collecting fluids (e.g., saline or other preparations) and the like.

In conventional endoscopy, the distal portion of the endoscope can be configured for supporting and orienting a therapeutic device, such as with the use of an elevator. In some systems, two endoscopes can be configured to work together with a first endoscope guiding a second endoscope inserted therein with the aid of the elevator. Such systems can be helpful in guiding endoscopes to anatomic locations within the body that are difficult to reach. For example, some anatomic locations can only be accessed with an endoscope after insertion through a circuitous path. For example, duodenoscopy procedures (e.g., Endoscopic Retrograde Cholangio-Pancreatography, hereinafter “ERCP” procedures) involve the use of an auxiliary scope (also referred to as a daughter scope or cholangioscope) that can be advanced through the working channel of a main scope (also referred to as a mother scope or duodenoscope). Furthermore, another device, such as a tissue retrieval device used for biopsies, can be inserted into the auxiliary scope. Typically, a duodenoscope, auxiliary scope and tissue retrieval device become progressively smaller since such scopes are configured in telescoping arrangements. Typically, after each use, the duodenoscope, auxiliary scope and tissue retrieval device are cleaned and sterilized for reuse. As such, imaging and control systems have light generators, image processing capabilities and treatment functionality are typically configured for repeated use with the same type or same types of endoscopes and instruments.

### Overview

The present disclosure recognizes that problems to be solved with surgical systems involve the need to adapt disposable endoscopes for use with existing imaging and control systems. There has been a recent desire to utilize disposable endoscopes to, for example, eliminate the need to clean, sterilize and reprocess reusable scopes. However, much capital equipment, such as light generators, image processing equipment and treatment equipment, is configured for use with reusable endoscopes having specific compatibilities, such as lighting and imaging system compatibility. In particular, many endoscopes include light transmitting capabilities, such as light conductors or light pipes, that transmit light generated at the imaging and control system to the distal end of the endoscope for use in the anatomy. As such, the operator of the endoscope can control the imaging and lighting features of the endoscope from the imaging and control system. Thus, there is a need to produce disposable endoscopes that are both compatible with existing imaging and control systems and that are inexpensive.

The present disclosure also recognizes that problems to be solved with disposable surgical systems involve the need to reproduce, not only the compatibility, but the output and usability of conventional endoscopes in disposable endoscopes. Sometimes, it is desirable to use less costly components in single-use or disposable endoscopes to mitigate the expense of the disposable endoscope. However, less expensive components can perform in a different manner than components used in reusable endoscopes, where the expense of added performance is justified by multiple uses. The less expensive components desirable for single use endoscopes, can, however, produce different outputs than the more expensive components.

The present disclosure can provide solutions to these and other problems by providing systems, devices and methods relating to adapters that can transmit imaging instructions between and imaging and control system and an endoscope, particularly a disposable endoscope having components selected for coast and disposability. For example, reusable endoscopes can include imaging components, e.g., cameras, sensors or microchips, configured to provide a video feed to the imaging and control system. Sometimes, these imaging components can operate in conjunction with fisheye lenses that provide a deliberately distorted image combining a wide-angle view at the periphery of the image and a magnified view at the center of the image. Such imaging provides a surgeon both a magnified view of the tissue directly in front of the camera, which can be useful in identifying target anatomy, and a wide-angle view surrounding the magnified image, which can be useful in navigating through the anatomy and providing context to the magnified image. However, fisheye lenses add an additional cost to the endoscope and take up space within the endoscope. Thus, it can be desirable to omit such a feature from a disposable endoscope to reduce cost and save space. The present disclosure allows for images generated by a disposable endoscope to be transformed or processed before transmission to the imaging and control system. Such transformed or processed images can provide output that is familiar to users of reusable endoscopes and that can be customized. In particular, an image processing adapter for a disposable endoscope can be configured to obtain flat imaging signals from a camera that does not produce fisheye images and electronically distort the image to reproduce a fisheye output for the imaging and control system. Additionally, image processing adapters of the present disclosure can provide other image processing capabilities, such as zoom, crop, stretch, warp, distort, scale, skew, interpolate and other image processing. Image processing adapters of the present disclosure can also provide image shifting capabilities where pixels of a raw digital image having a certain resolution can be cropped to have less resolution and shifted, e.g., reframed, to be taken offset from a center of the raw digital image to, for example, focus on a more desirable portion, e.g., an unobstructed portion, of the image or a portion including an area of interest. Furthermore, image processing adapters of the present disclosure can produce three-dimensional (3D) images from a sequence of images.

In an example, an adapter for an endoscope system can comprise a housing, an input device for receiving an imaging signal input from an endoscope, an imaging signal processor configured to receive the imaging signal input and perform an image processing technique to the imaging signal input to generate an imaging signal output, the imaging signal processing technique configured to alter a display characteristic of the imaging signal input, and an output coupler for providing the imaging signal output to an imaging and control system.

In another example, a surgical endoscope system can comprise an imaging and control system comprising a socket, an endoscope comprising a shaft comprising an imaging device at a distal end portion a working channel extending at least partially through the shaft and an imaging sensor at the distal end portion, the imaging sensor configured to generate an endoscope imaging signal including a flat image, a communication device for transmitting output of the imaging sensor, and an adapter configured to be connected to the socket, the adapter comprising an input device configured to communicate with the communication device of the endoscope to receive the endoscope imaging signal, a converter configured to transform the flat image of the endoscope imaging signal into a transformed image of an output imaging signal and an output coupler configured to connect to the socket of the imaging and control system to convey the output imaging signal.

In an additional example, a method for transforming a flat image to a distorted image can comprise mapping pixels of the flat image to a first table correlating the pixels to locations in the flat image, correlating the locations of the first table to locations in a second table for the distorted image, and generating the distorted image by moving pixels from the locations of the first table to associated locations in the second table.

### BRIEF DESCRIPTION OF DRAWINGS

FIG. 1 is a schematic diagram of an endoscopy system comprising an imaging and control system and an endoscope, such as duodenoscope, with which the image processing adapters of the present disclosure can be used.

FIG. 2 is a schematic diagram of the endoscopy system of FIG. 1 showing the imaging and control system connected to the endoscope.

FIG. 3 is a block diagram showing a light-conducting and signal-relaying connector coupling the imaging and control system and endoscope of FIGS. 1 and 2.

FIG. 4 is a block diagram showing an image processing adapter of the present disclosure coupling the connector of the imaging and control system of FIGS. 1 and 2 to an endoscope having an on-board image generator.

FIG. 5 is a rear perspective view of an adapter of the present disclosure showing connections for coupling to an imaging and control system.

FIG. 6 is a front perspective view of the adapter of FIG. 5 showing a socket for coupling to an endoscope.

FIG. 7 is a rear end view of the adapter of FIGS. 5 and 6 showing a light conductor and an air coupler for connecting to an imaging and control system.

FIG. 8 is a front end view of the adapter of FIGS. 5 and 6 showing an electronics port, an air port and alignment posts for connecting to an endoscope.

FIG. 9 is a front perspective view of the adapter of FIGS. 5-8 with an outer housing removed to show a support bracket connected to a plug component and a socket component.

FIG. 10 is a front perspective view of the adapter of FIG. 9 with the support bracket and socket component removed to show a light conductor, circuit boards, a communications port, an air tube and an air outlet.

FIG. 11 is a cross-sectional view through the adapter of FIG. 10 showing an air passage through the adapter and image processing circuitry positioned electrically between a communications port and electrical leads.

FIG. 12 is a block diagram illustrating an example of an image processing adapter of the present disclosure including image processing circuitry.

FIG. 13 is a schematic diagram illustrating an image processing adapter converting a flat image obtained from an endoscope imaging sensor to a fisheye image for use at an imaging and control system.

FIG. 14A is a schematic diagram of a flat image being transformed to a fisheye image via an image manipulation process.

FIG. 14B is a schematic diagram of pixels of the flat digital image of FIG. 14A being transformed to the fisheye image of FIG. 14A.

FIG. 15A is a schematic diagram of a flat image being transformed to a pincushion image via an image manipulation process.

FIG. 15B is a schematic diagram of pixels of the flat digital image of FIG. 15A being transformed to the pincushion image of FIG. 15A.

FIG. 16 is a block diagram illustrating operations of methods for converting imaging signals generated by an endoscope to processed imaging signals generated by an image processing adapter of the present disclosure.

FIG. 17A is a diagram illustrating a digital image from an imaging sensor showing the presence of a medical instrument component in the field of view and a processed image boundary on the digital image.

FIG. 17B is a diagram illustrating the digital image of FIG. 17B with the processed image boundary shifted to adjust the presence of the medical instrument component.

In the drawings, which are not necessarily drawn to scale, like numerals may describe similar components in different views. Like numerals having different letter suffixes may represent different instances of similar components. The drawings illustrate generally, by way of example, but not by way of limitation, various embodiments discussed in the present document.

### DETAILED DESCRIPTION

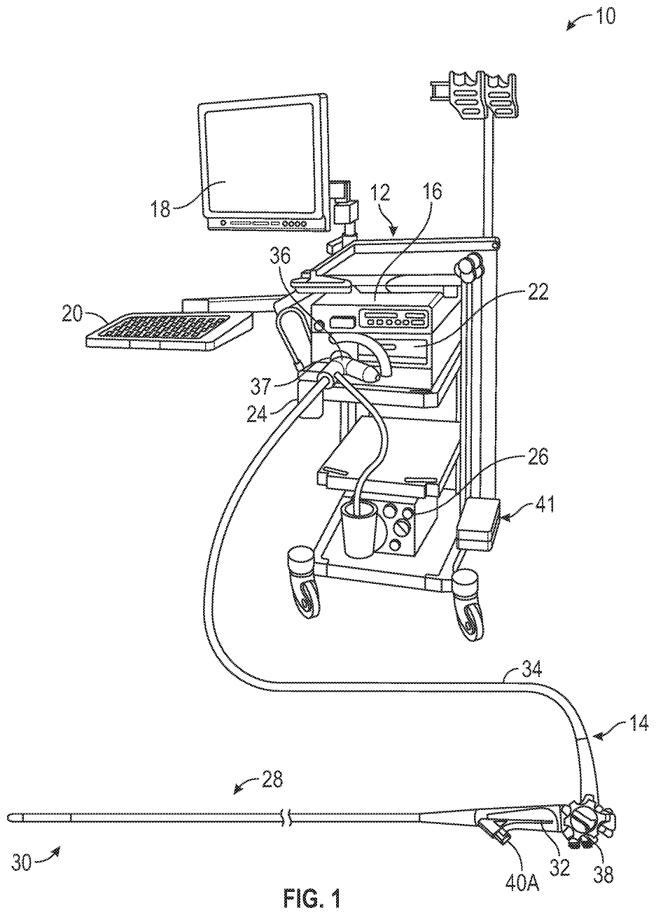

FIG. 1 is a schematic diagram of endoscopy system 10 comprising imaging and control system 12 and endoscope 14. The system of FIG. 1 is an illustrative example of an endoscopy system suitable for use with the systems, devices and methods described herein, such as image processing adapters. According to some examples, endoscope 14 can be insertable into an anatomical region for imaging and/or to provide passage of other devices, such as auxiliary scopes and biopsy devices or one or more therapeutic devices for treatment of a disease state associated with the anatomical region. Endoscope 14 can, in advantageous aspects, interface with and connect to imaging and control system 12 such as via insertion of coupler section 36 into socket 37. In the illustrated example, endoscope 14 comprises a duodenoscope, though other types of endoscopes can be used with the features and teachings of the present disclosure.

Imaging and control system 12 can comprise control unit 16, output unit 18, input unit 20, light source unit 22, fluid source 24 and suction pump 26.

Imaging and control system 12 can include various ports for coupling with endoscopy system 10. For example, control unit 16 can include a data input/output port for receiving data from and communicating data to endoscope 14. Such data input/output can be provided through an interface between coupler section 36 and socket 37. Light source unit 22 can include an output port for transmitting light to endoscope 14, such as via a fiber optic link.

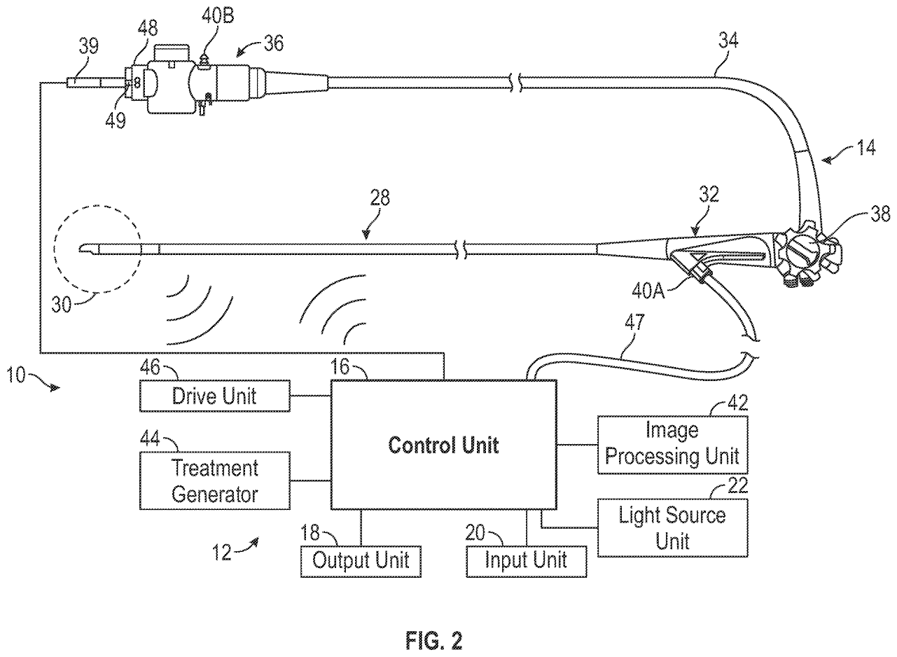

For example, coupler section 36 can include light conductor 39 (FIG. 2) that is configured to receive light from a lens or bulb within light source unit 22. Fluid source 24 can include a port for transmitting fluid to endoscope 14. Fluid source 24 can comprise a pump and a tank of fluid or can be connected to an external tank, vessel or storage unit. Suction pump 26 can comprise a port used to draw a vacuum from endoscope 14 to generate suction, such as for withdrawing fluid from the anatomical region into which endoscope 14 is inserted. In examples, fluid, such as air, can be transferred to endoscope 14 through an interface at coupler section 36 and socket 37. In examples, fluids, such as water, can be directly input into coupler section 36 without emanating from socket 37. Output unit 18, e.g., a touch-screen display, and input unit 20, e.g., a keyboard, can be used by an operator of endoscopy system 10 to control functions of endoscopy system 10 and view output of endoscope 14. Control unit 16 can additionally be used to generate signals or other outputs from treating the anatomical region into which endoscope 14 is inserted. In examples, control unit 16 can generate electrical output, acoustic output, a fluid output and the like for treating the anatomical region with, for example, cauterizing, cutting, freezing and the like.

Endoscope 14 can comprise insertion section 28, functional section 30 and handle section 32, which can be coupled to cable section 34 and coupler section 36. Coupler section 36 can be connected to control unit 16 at socket 37 to connect to endoscope 14 to multiple features of control unit 16, such as input unit 20 and light source unit 22. Fluid source 24 and suction pump 26 can be connected directly to endoscope 14 without routing through control unit 16.

Insertion section 28 can extend distally from handle section 32 and cable section 34 can extend proximally from handle section 32. Insertion section 28 can be elongated and include a bending section, and a distal end to which functional section 30 can be attached. The bending section can be controllable (e.g., by control knob 38 on handle section 32) to maneuver the distal end through tortuous anatomical passageways (e.g., stomach, duodenum, kidney, ureter, etc.). Insertion section 28 can also include one or more working channels (e.g., an internal lumen) that can be elongate and support insertion of one or more therapeutic tools of functional section 30, such as an auxiliary scope. The working channel can extend between handle section 32 and functional section 30. Additional functionalities, such as fluid passages, guide wires, and pull wires can also be provided by insertion section 28 (e.g., via suction or irrigation passageways, and the like).

Handle section 32 can comprise control knob 38 as well as port 40A. Control knob 38 can be coupled to a pull wire, or other actuation mechanisms, extending through insertion section 28. Port 40A, as well as other ports, such as port 40B (FIG. 2), can be configured to couple various electrical cables, guide wires, auxiliary scopes, tissue collection devices, fluid tubes and the like to handle section 32 for coupling with insertion section 28. In examples, handle section 32 can additionally include control features for operating capabilities of functional section 30 and image processing adapters of the present disclosure.

Imaging and control system 12, according to examples, can be provided on a mobile platform (e.g., cart 41) with shelves for housing light source unit 22, suction pump 26, image processing unit 42 (FIG. 2), etc. Alternatively, several components of imaging and control system 12 shown in FIGS. 1 and 2 can be provided directly on endoscope 14 so as to make the endoscope “self-contained.”

Functional section 30 can comprise components for treating and diagnosing anatomy of a patient. Functional section 30 can comprise an imaging device, an illumination device (e.g., the distal end of a light fiber) and an elevator. In examples, the imaging device can be used with various lenses, such as lenses that can bend light or protect the imaging device. Operation of some or all features of functional section 30 is typically performed at imaging and control system 12 or at handle section 32.

FIG. 2 is a schematic diagram of endoscopy system 10 of FIG. 1 comprising imaging and control system 12 and endoscope 14. FIG. 2 schematically illustrates components of imaging and control system 12 coupled to endoscope 14, which in the illustrated example comprises a duodenoscope. Imaging and control system 12 can comprise control unit 16, which can include or be coupled to image processing unit 42, treatment generator 44 and drive unit 46, as well as light source unit 22, input unit 20 and output unit 18. Coupler section 36 can be connected to control unit 16 to connect to endoscope 14 to multiple features of control unit 16, such as image processing unit 42 and treatment generator 44. In examples, plug portion 48 of coupler section 36 can include leads 49 for connecting to wiring within socket 37 (FIG. 1) that can connect to one or more of light source unit 22, imaging processing unit 42 and treatment generator 44. In examples, port 40A can be used to insert another instrument or device, such as a daughter scope or auxiliary scope, into endoscope 14. Such instruments and devices can be independently connected to control unit 16 via cable 47 or can extend directly from fluid source 24 and suction pump 26 without coming from control unit 16. In examples, port 40B can be used to connect coupler section 36 to various inputs and outputs, such as video, air, light and electric. Control unit 16 can be configured to activate a camera to view target tissue distal of endoscope 14. In examples, functional section 30 can include an imaging device that can transmit an imaging signal to imaging and control system 12 through leads 49 that connect to wiring within socket 37 (FIG. 1). Likewise, control unit 16 can be configured to activate light source unit 22 to direct light into endoscope 14 or other devices extending therefrom. Light source unit 22 can comprise a light generator, such as a xenon bulb or a light emitting diode. In example, light source unit 22 can include multiple light generators to generate light with different properties, such as different color.

Image processing unit 42 and light source unit 22 can each interface with endoscope 14 (e.g., at functional section 30) by wired or wireless electrical connections. Imaging and control system 12 can accordingly illuminate an anatomical region, collect signals representing the anatomical region, process signals representing the anatomical region, and display images representing the anatomical region on output unit 18. Imaging and control system 12 can include light source unit 22 to illuminate the anatomical region using light of desired spectrum (e.g., broadband white light, narrow-band imaging using preferred electromagnetic wavelengths, and the like). Imaging and control system 12 can connect (e.g., via an endoscope connector or socket 37 (FIG. 1)) to endoscope 14 for signal transmission (e.g., light output from light source, video signals from imaging system in the distal end, diagnostic and sensor signals from a diagnostic device, and the like).

Fluid source 24 (FIG. 1) can be in communication with control unit 16 and can comprise one or more sources of air, saline or other fluids, as well as associated fluid pathways (e.g., air channels, irrigation channels, suction channels) and connectors (barb fittings, fluid seals, valves and the like). Fluid source 24 can be utilized as an activation energy for a biasing device or a pressure-applying device. Imaging and control system 12 can also include drive unit 46, which can be an optional component. Drive unit 46 can comprise a motorized drive for advancing a distal section of endoscope 14, as described in at least PCT Pub. No. WO 2011/140118 A1 to Frassica et al., titled “Rotate-to-Advance Catheterization System,” which is hereby incorporated in its entirety by this reference.

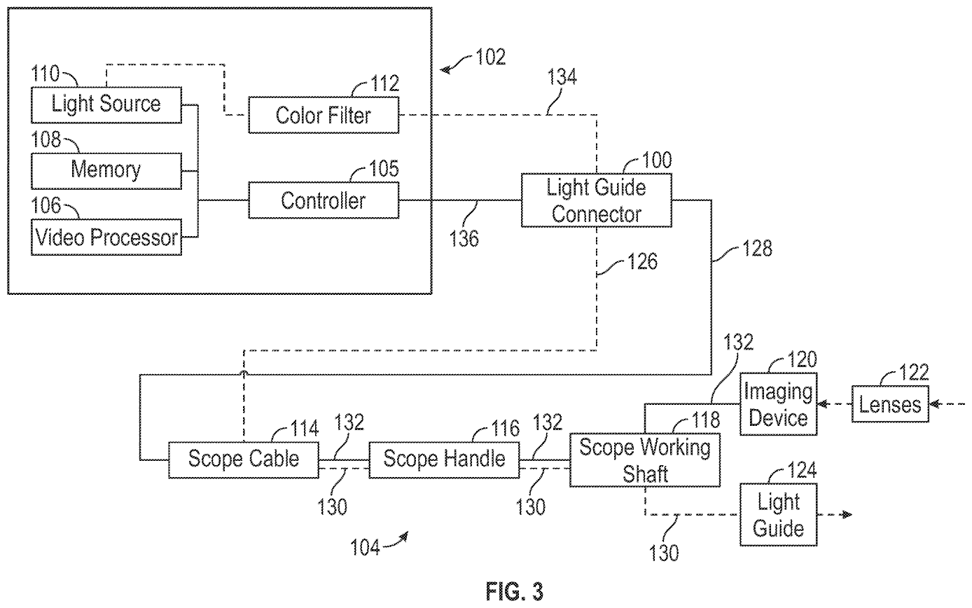

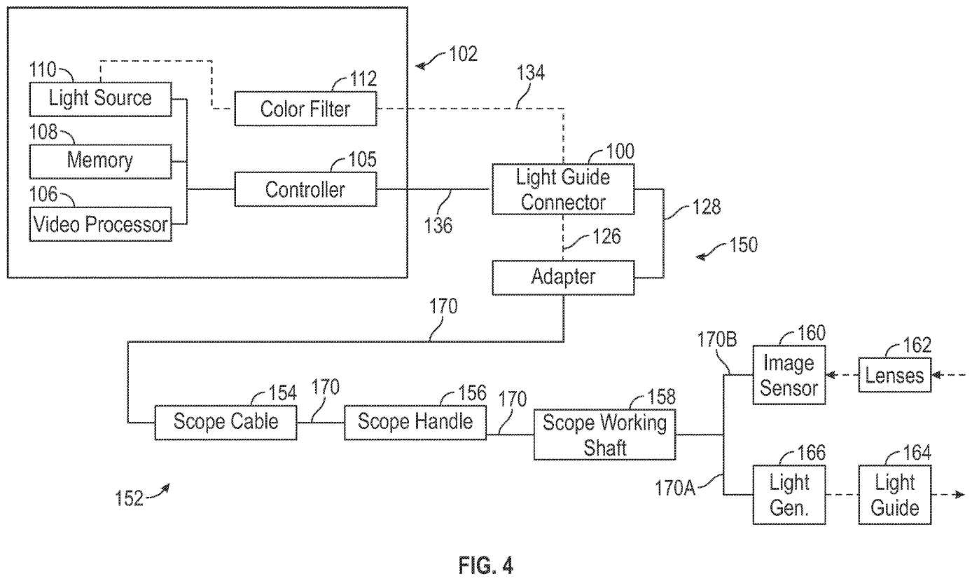

As mentioned, coupler section 36 can be used to connected endoscope 14 with imaging and control system 12. Coupler section 36 can be used to communicate various functions between endoscope 14 and imaging and control system 12. In examples, coupler section 36 can transmit communication signals, electronic signals, electrical signals, power signals, fluids including water and air, light waves and the like. Coupler section 36 can comprise a part of endoscope 14 and can be configured for particular configurations of imaging and control system 12. For example, coupler section 36 can be configured to transmit light generated by light source unit 22 to endoscope 14 using light conductor 39, as is discussed with reference to FIG. 3. Additionally, coupler section 36 can utilize leads 49 to transmit imaging signals between functional section 30 and imaging and control system 12. With the present disclosure, an image processing adapter can be connected to imaging and control system 12 to couple to an endoscope having built-in or on-board image processing capabilities, as is discussed in greater detail with reference to FIG. 4. The image processing adapters can connect imaging and control system 12 to an endoscope via wired or wireless connections. Such image processing adapters can convert, transform, shift or otherwise manipulate imaging signals generated by an imaging sensor of the endoscope to another image output, such as image shape or image format, for display to a user on output unit 18 (FIG. 1). Thus, for example, the output of a disposable endoscope having a less expensive imaging sensor can be electronically transformed to replicate the output of more expensive imaging sensors and to reproduce images familiar to users of non-disposable or reusable endoscopes. In examples, the image processing adapters can convert flat images, to images having a fisheye distortion (e.g., a barrel distortion), or a pincushion distortion, or to images that are cropped, distorted, skewed, stretched, scaled warped, zoomed, interpolated and the like, as discussed in greater detail below. Image processing adapters of the present disclosure can additionally crop and shift raw digital images to use only a portion of the raw digital image that may be offset from a center of the raw digital image, e.g., asymmetrically cropped, thereby showing a portion of the raw digital image having more features of intertest. Additionally, image processing adapters of the present disclosure can combine multiple two-dimensional images to form three-dimensional images.

FIG. 3 is a block diagram showing light guide connector 100 coupling imaging and control system 102 to endoscope 104. Imaging and control system 102 can comprise an instance of imaging and control system 12 of FIGS. 1 and 2. Imaging and control system 102 can comprise controller 105, video processor 106, memory 108, light source 110 and filter 112. In examples, controller 105 can comprise an instance of control unit 16 of FIG. 2, light source 110 can comprise an instance of light source unit 22 of FIG. 2, and video processor 106 can comprise an instance of image processing unit 42 of FIG. 2. Endoscope 104 can comprise scope cable 114, scope handle 116, scope working shaft 118, imaging device 120, lens 122 and light guide 124. In examples, scope cable 114 can comprise an instance of cable section 34 of FIG. 2, scope handle 116 can comprise an instance of handle section 32 of FIG. 2, and scope working shaft 118 can comprise an instance of insertion section 28 of FIG. 2. In examples, light guide connector 100 can comprise an instance of socket 37 of FIG. 1. As such, scope cable 114 can comprise a coupler similar to coupler section 36 of FIG. 1.

Light guide connector 100 can be used to convey electronic signals, e.g., imaging signals, and light waves between imaging and control system 102 and endoscope 104. In FIG. 3, light waves can be indicated by dashed lines and wired signals can be indicated by solid lines. Light guide connector 100 can transmit electronic signals generated by imaging device 120 to imaging and control system 102 and control signals from controller 105 to endoscope 104. For example, control signals for operating various features of endoscope 104, such as ablation, suturing, RF signal generation, cryogenic features and the like, can be conveyed from controller 105 to endoscope 104. Furthermore, light waves from light source 110 can be conveyed to endoscope 104 via light guide connector 100.

Light guide connector 100 can include light conductor 126 and electric wiring 128. Endoscope 103 can include light conductor 130 and electric wiring 132. Imaging and control system 102 can include light conductor 134 and control wiring 136. Light conductor 126 of light guide connector 100 can connect light conductor 130 of endoscope 104 to light source 110 via light conductor 134, and electric wiring 128 of light guide connector 100 can connect electric wiring 132 of endoscope 104 to controller 105 via control wiring 136.

Endoscope 104 can control transmission of electronic imaging signals from imaging device 120 to imaging and control system 102. For example, light can enter lens 122 at endoscope 104. Lens 122 can bend or distort light waves passing therethrough to distort or otherwise transform the image generated by imaging device 120. In examples, lens 122 can comprise a fisheye lens or other customized lenses to produce desirable output images. Lens 122 can be used to enhance the input to imaging device 120 to generate magnified or desirably distorted images, such as fisheye distortions or pincushion distortions. The light can be received by imaging device 120. In examples, imaging device 120 can comprise a charge-coupled device (CCD) or a solid state device such as a complementary metal oxide semiconductor (CMOS). Imaging device 120 can convert the light waves received from lens 122 to electronic signals. The electronic signals can be passed through scope working shaft 118, scope handle 116 and scope cable 114 via appropriate conductors of electric wiring 132 to electric wiring 128 of light guide connector 100. Electric wiring 128 of light guide connector 100 can include appropriate couplers for transmitting the electronic signal from imaging device 120 to imaging and control system 102 through control wiring 136. As such, video processor 106 can receive the electronic signals from imaging device 120 for displaying on a video monitor, such as output unit 18 of FIG. 1, after appropriate processing and the like. Memory 108 can include various red, green and blue image memories for processing signals generated by imaging device 120. As such, with typical reusable endoscopes, the image data or signal generated by imaging device 120 can be relayed from endoscope 104 to imaging and control system 102 through light guide connector 100 without alteration.

In addition to light signals and imaging signals, light guide connector 100 can relay other types of data, such as control signals for various functions of endoscope 104. In particular, control wiring 136, electric wiring 128 and electric wiring 132 can additionally be used to convey control signals for diagnostic and treatment functionality of endoscope 104. For example, a user can input setting for functionality of endoscope 104 in controller 105 using, for example, input unit 20 (FIG. 2). Controller 105 can then generate appropriate control signals for transmission to light guide connector 100. Electric wiring 128 can be configured to carry control signals with additional conductors or the same conductors that carry the imaging signals using, for example, leads 49 (FIG. 2).

Furthermore, though not illustrated in FIG. 3, light guide connector 100 can include appropriate tubing or piping to carry fluids, such as saline, irrigation fluid, air, insufflation gas and other gases and the like, to and from endoscope 104.

Light source 110 can control the intensity and type of light generated by imaging and control system 102. For example, light source 110 or features of imaging and control system 102, such as input unit 20 (FIG. 1), can include control features, such as buttons or knobs to start and stop generation of light waves, control the intensity of the light waves and the like. Imaging and control system 102 can additionally include control features for activating or deactivating different types of filters of filter 112, such as color filters and the like. Thus, a user of imaging and control system 102 can initiate settings for light to be transmitted to light guide 124 of endoscope 104 at imaging and control system 102. Typical user settings include: 1) on/off, 2) light intensity, and 3) light color. In examples, 1) the on/off setting can be a function of intensity (e.g., zero intensity equals off), 2) the intensity setting can be a function of current or electrical signal provided to light source 110, and 3) the color setting can be a function of which of filters 112 is applied to output of light source 110. Each of 1), 2) and 3) can be set by a user at controller 105 and can be indicated as a property of light waves emitted from light source 110. Light conductor 126 of light guide connector 100 can include appropriate couplers, conductors or pipes for transmitting light waves from light conductor 134 of light source 110 to light guide 124. As such, light waves from light source 110 can travel through filter 112, light conductor 134 of imaging and control system 12, light conductor 126 of light guide connector 100, light conductor 130 of endoscope 104 (including light conductor 39 of FIG. 2), and light guide 124, whereby the light waves can exit endoscope 14 to illuminate anatomy into which endoscope 104 is inserted.

Configured as such, light guide connector 100 can be configured to relay signals and light waves between imaging and control system 102 and endoscope 104 without modification. In examples, endoscope 104 can be specifically configured for operation with imaging and control system 102. For example, light guide 124 can be configured to transmit light waves generated by light source 110 without interruption or introducing any distortions, such as discolorations or intensity changes. Additionally, light guide connector 100 can provide an electronic communications pathway between imaging device 120 and video processor 106 and between controller 105 and functionality of endoscope 104. As such, light guide connector 100 does not include any capability for interpreting, analyzing or changing light signals, imaging signals and control signals. Furthermore, light guide connector 100 can be mechanically configured to couple to particular types of endoscope plugs, such as coupler section 36 of FIG. 1. Thus, other types of endoscopes not configured to receive the outputs of imaging and control system 12 or that are not mechanically configured to mate with socket 37 are not inter-operable or compatible with imaging and control system 12.

FIG. 4 is a block diagram showing adapter 150 of the present disclosure coupling imaging and control system 102 of FIGS. 1 and 2 to endoscope 152.

Imaging and control system 102 can comprise controller 105, video processor 106, memory 108, light source 110 and filter 112. Imaging and control system 102 can be configured similarly as described with reference to FIG. 3 to provide light output at light conductor 134 and to send and receive communication signals via control wiring 136.

Endoscope 152 can comprise scope cable 154, scope handle 156, scope working shaft 158, imaging device 160, lens 162, light guide 164 and light generator 166. Endoscope 152 can be configured similarly as endoscope 104 of FIG. 3, except that rather than endoscope 152 having light conductor 130 extending therethrough as in endoscope 104, endoscope 152 can include light generator 166. In examples, scope cable 154 can be configured similarly as cable section 34 of FIG. 2, scope handle 156 can be configured similarly as handle section 32 of FIG. 2, and scope working shaft 158 can be configured similarly as insertion section 28 of FIG. 2, with the inclusion of imaging device 160 instead of a light conductor proximal thereof. In examples, light guide connector 100 can comprise an instance of socket 37 of FIG. 1.

Adapter 150 can be used to convey information from light guide connector 100 to endoscope 152. Adapter 150 can be configured for insertion into socket 37 (FIG. 1) to receive light from light source unit 22 and control signals from control unit 16, as well as various air sources. Adapter 150 can be configured to receive imaging signals from imaging device 160, perform imaging processing techniques to the imaging signals, and transmit the processed imaging signals to imaging and controls system 102. Light guide connector 100 can be used to convey electronic signals and light waves between imaging and control system 102 and adapter 150. In FIG. 4, light waves can be indicated by dashed lines and wired signals can be indicated by solid lines. Adapter 150 can transmit electronic signals generated by imaging device 120 to light guide connector 100 for transmission to imaging and control system 102 after performing image processing or, optionally, not performing image processing. Adapter 150 can additionally transmit electronic signals from controller 105 and light guide connector 100 to endoscope 152. Adapter 150 can receive light waves from light guide connector 100 generated by light source 110 and can convert such light waves into combined signal wiring 170 for transmission to light generator 166. Light generator 166 can comprise a light source configured to output light waves. In examples, light generator 166 can comprise a light emitting diode (LED). In additional examples, light generator 166 can be configured to generate light of different colors.

Adapter 150 can include combined signal wiring 170, which can extend through endoscope 152. Combined signal wiring 170 can branch into light signal wiring 170A for communicating with light generator 166 and imaging signal wiring 170B for communicating with imaging device 160. Imaging and control system 102 can include light conductor 134 and control wiring 136. Light conductor 126 and electric wiring 128 of light guide connector 100 can connect to adapter 150 and adapter 150 can transmit combined signal wiring 170 to light generator 166 and imaging device 160. Combined signal wiring 170, light signal wiring 170A and imaging signal wiring 170B are depicted in the illustrated example of FIG. 4 as comprising wired connections. However, in various examples, any or all of combined signal wiring 170, light signal wiring 170A and imaging signal wiring 170B can comprise a wireless signal. For example, scope working shaft 158 or scope handle 156 can include a wireless transmitter to receive signals from light signal wiring 170A and imaging signal wiring 170B and convert those signals to wireless signal for transmission to a wireless antenna at adapter 150. Endoscope 152 and adapter 150 can include wireless hardware to allow for two-way wireless communication between endoscope 152 and adapter 150. In additional examples, endoscope 152 can communicate with imaging and control system 102 via wireless communication. In examples where a wireless transmitter is included in endoscope 152, scope cable 154 need not extend to and connect with adapter 150. However, redundant communication systems can be provided wherein endoscope 152 and adapter 150 are configured to communicate with imaging and control system 12 via wired and wireless means.

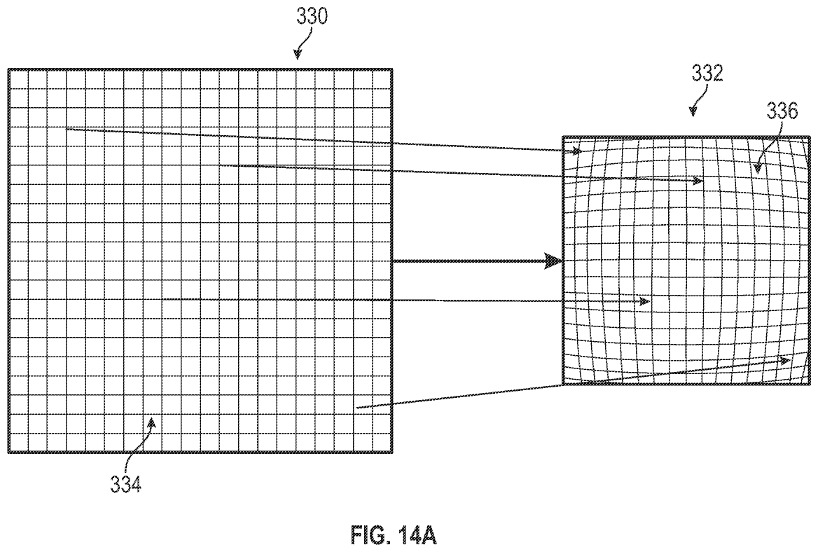

Endoscope 152 can control transmission of electronic imaging signals from imaging device 160 to imaging and control system 102. For example, light can enter lens 162 at endoscope 152. In examples, lens 162 can be omitted because, for example, lens 162 can add cost and take up space. In examples, lens 162 can comprise a protective lens that does not bend or transform light passing through it. However, light bending lenses can be used in some examples. The light can be received by imaging device 160. In examples, imaging device 160 can comprise a charge-coupled device (CCD) or a solid state device such as a complementary metal oxide semiconductor (CMOS). Imaging device 160 can convert the light waves received from lens 162 to electronic signals. The electronic signals can be passed through scope working shaft 158, scope handle 156 and scope cable 154 via appropriate conductors of combined signal wiring 170 to electric wiring 128 of light guide connector 100 via adapter 150. As described in greater detail below, such as with reference to FIG. 12, adapter 150 can include circuitry, such as memory and processors, to transform the raw output of imaging device 160 into a transformed image output that can include one more of zooming, cropping, shifting, reframing, stretching, warping, distorting, scaling, skewing, interpolating and other image processing techniques, including three-dimensional picture generation. Electric wiring 128 of light guide connector 100 can include appropriate couplers for transmitting the electronic signal from adapter 150 to imaging and control system 102 through control wiring 136. As such, video processor 106 can receive processed imaging signals the electronic signals that originated from imaging device 160 for displaying on a video monitor, such as output unit 18 of FIG. 1, after appropriate filtering and the like.

Light source 110 can control the intensity and type of light generated by imaging and control system 102, as explained above. For example, light source 110 or features of imaging and control system 102, such as input unit 20 (FIG. 1), can include control features, such as buttons or knobs to start and stop generation of light waves, control the intensity of the light waves and the like, to control 1) on/off of light source 110, 2) intensity of light from light source 11, and 3) color of light as determined by filters 112. Light conductor 126 of light guide connector 100 can include appropriate couplers, conductors or pipes for transmitting light waves from light conductor 134 of light source 110 to adapter 150. Adapter 150 can receive light waves from light conductor 134 and convert sensed properties, e.g., on/off, intensity and color, of the light waves into electronic control signal for light generator 166. Adapter 150 can include appropriate sensors and circuitry to convert light waves into electronic control signals, as is discussed herein. As such, light waves from light source 110 can travel through filter 112, light conductor 126 of light guide connector 100 to adapter 150, followed by adapter 150 transmitting light generation signals along combined signal wiring 170 to light generator 166, which can thereafter output light to light guide 164, whereby the light waves can exit endoscope 152 to illuminate anatomy into which endoscope 152 is inserted. Thus, when an operator of imaging and control system 102 calls for light, e.g., instructs light source 110 to be on, light generator 166 can be commanded to generate light waves equivalent in intensity and color as light waves exiting light source 110.

Configured as such, adapter 150 can be configured to relay signals between imaging and control system 102 and endoscope 152 with translation, modification or interpolation. Endoscope 152 need not be specifically designed to operate with imaging and control system 102 and can include any type of light generator 166 and coupler section. In examples, endoscope 152 can be adapted for operation with imaging and control system 102 with the use of adapter 150. Adapter 150 can be configured to allow multiple different types of endoscopes to communicate with imaging and controls system 102. For example, adapter 150 can recognize the capabilities, e.g., a camera type, of an endoscope and can perform image processing functions to provide an image input to imaging and control system 12 that imaging and control system 12 is expecting to receive, thereby allowing different types of endoscopes to be compatible with imaging and control system 12. Adapter 150 can provide the appropriate mechanical interface between endoscope 152 and imaging and control system 102 and the appropriate translation of control inputs for 1), 2) and 3) entered at controller 105 to light generator 166. In addition to light signals, e.g., light waves, and imaging signal, e.g., electronic communication signals, adapter 150 can relay other types of data, e.g., control signals, as well as various fluids, such as water and air, between light guide connector 100 and endoscope 152. With the present disclosure, adapter 150 can additionally be configured to provide image processing capabilities as discussed herein. Adapter 150 can comprise a reusable part that is readily cleaned and sterilized, while endoscope 152 can be configured as a disposable scope that does not need to be cleaned or sanitized.

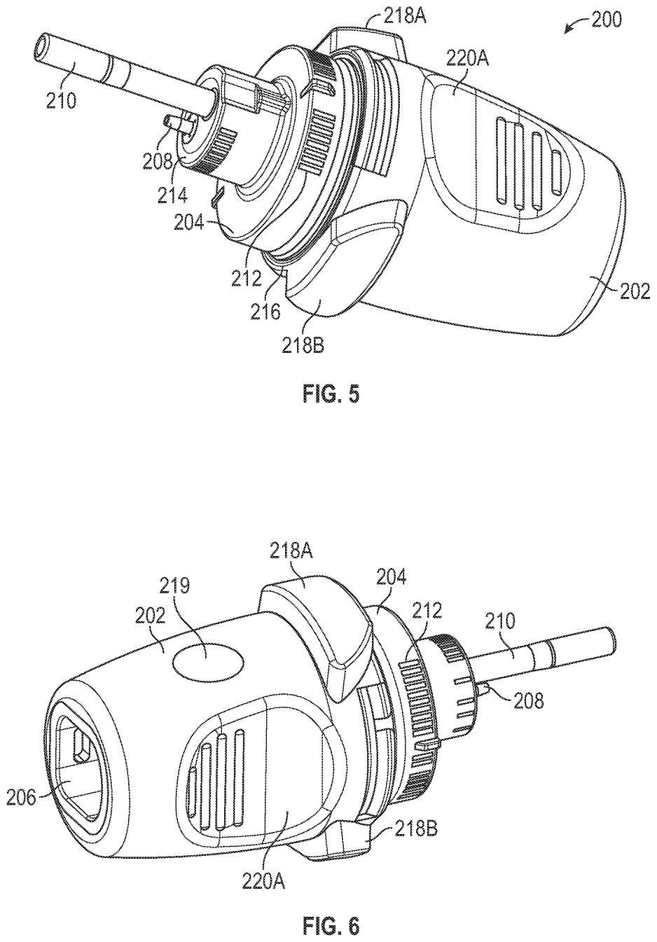

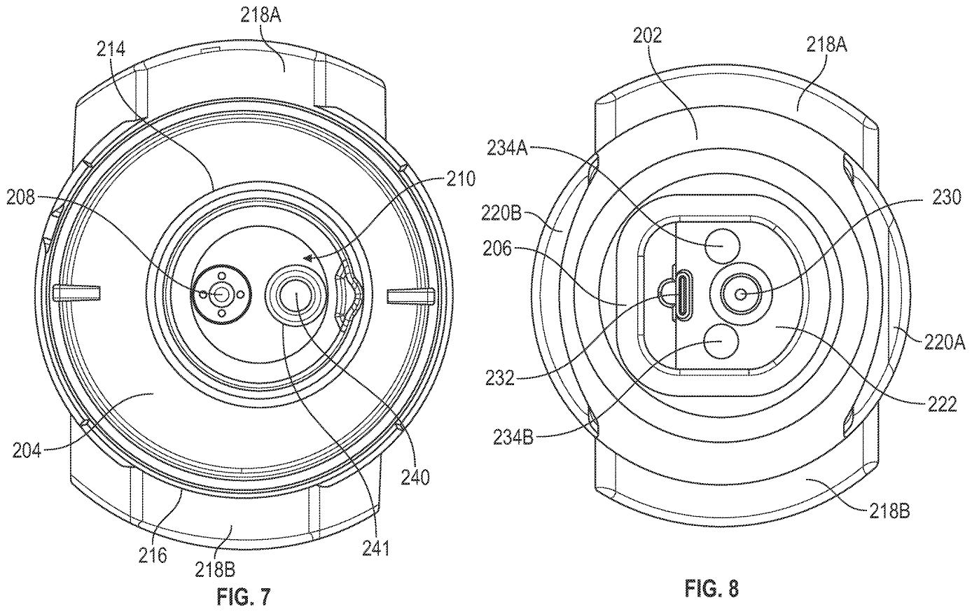

FIG. 5 is a rear perspective view of adapter 200 of the present disclosure showing main housing 202 and plug component 204. FIG. 6 is a front perspective view of adapter 200 of FIG. 5 showing socket component 206 for receiving an endoscope plug. Adapter 200 can comprise an instance of adapter 150 of FIG. 4. FIGS. 5 and 6 are discussed concurrently.

Plug component 204 can comprise air coupler 208, light conductor assembly 210 and electrical leads 212. Air coupler 208 and light conductor assembly 210 can extend from end face 214 of plug component 204. Electrical leads 212 can extend from shoulders or corners of plug component 204. Air coupler 208 and light conductor assembly 210 can be coupled to light guide connector 100 (FIG. 3) or directly to light source unit 22 (FIG. 1) or another component of imaging and control system 12. Plug component 204 can be inserted into receptacle 216 of main housing 202. Main housing 202 can comprise lugs 218A and 218B that can be coupled to a receptacle, e.g., socket 37 (FIG. 1) in light guide connector 100 or light source unit 22 via a twist-lock or push-pull operation. Adapter 200 can include button 219 to operate functions of adapter 200. In examples, button 219 can toggle the image processing features of the present disclosure, such as by enabling or disabling the image processing capabilities or cycling through different image processing capabilities. In examples, more than one button can be included to perform additional function, such as to allow for individual toggling on or off of different image processing functions. Main housing 202 can include other features such as pads 220A and 220B for providing ergonomic engagement with fingers of a user.

Adapter 200 can be configured to receive light waves at light conductor assembly 210, air at air coupler 208 and imaging and control signals at electrical leads 212 and 214. In examples, plug component 204 can be configured similarly as plug portion 48 of coupler section 36 (FIG. 2) and electrical leads 212 can operate similarly as lead 49. Thus, control signals generated by imaging and control system 12 (FIGS. 1 and 2) can be transmitted to adapter 200. Similarly, imaging signals generated by endoscope 152 (FIG. 4) can be transmitted to imaging and control system 12 via adapter 200. Light conductor assembly 210 can be configured similarly as light conductor 39 (FIG. 2). Thus, light output by light source unit 22 can be conveyed to adapter 200.

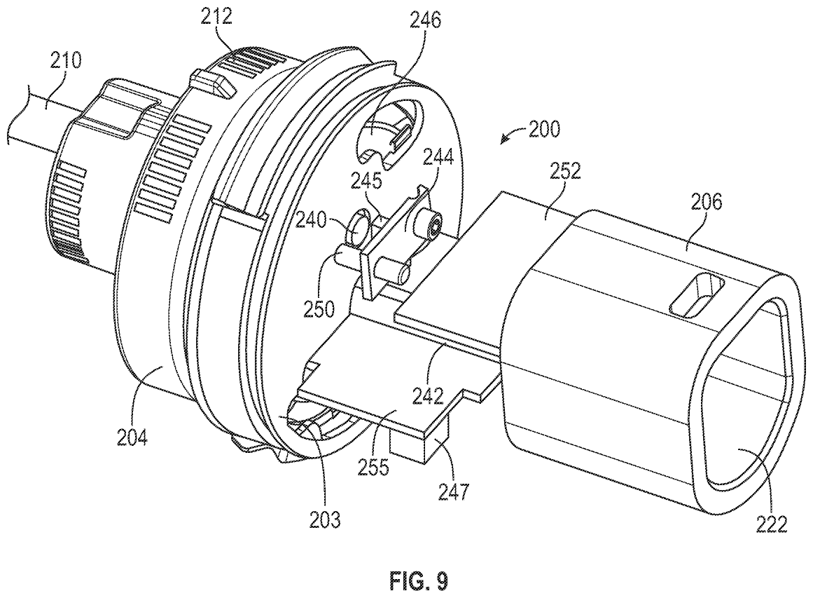

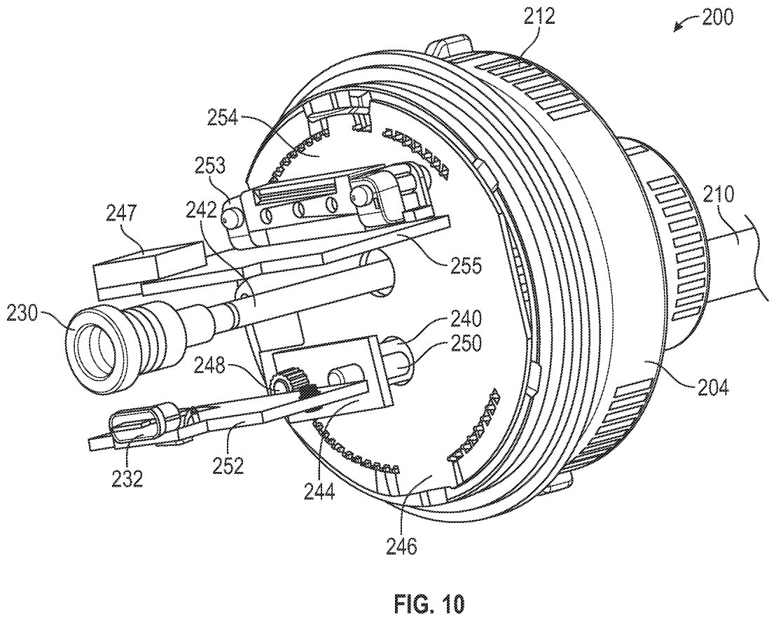

Socket component 206 can include opening 222 to receive an endoscope plug, such as a plug connected to combined signal wiring 170 (FIG. 4). Opening 222 can include air coupler 230 (FIG. 8) and electrical coupler 232 (FIG. 8) for communicating with endoscope 152 (FIG. 4). As discussed with reference to FIGS. 7 and 8, adapter 200 can allow air, imaging signals and control signals to pass through main housing 202 and plug component 204 via fluid coupler 230 and electrical coupler 232.

FIG. 7 is a rear end view of adapter 200 of FIGS. 5 and 6 showing light conductor assembly 210 and air coupler 208. FIG. 8 is a front end view of adapter 200 of FIGS. 5 and 6 showing air coupler 230, electrical coupler 232, and alignment posts 234A and 234B. FIGS. 7 and 8 are discussed concurrently.

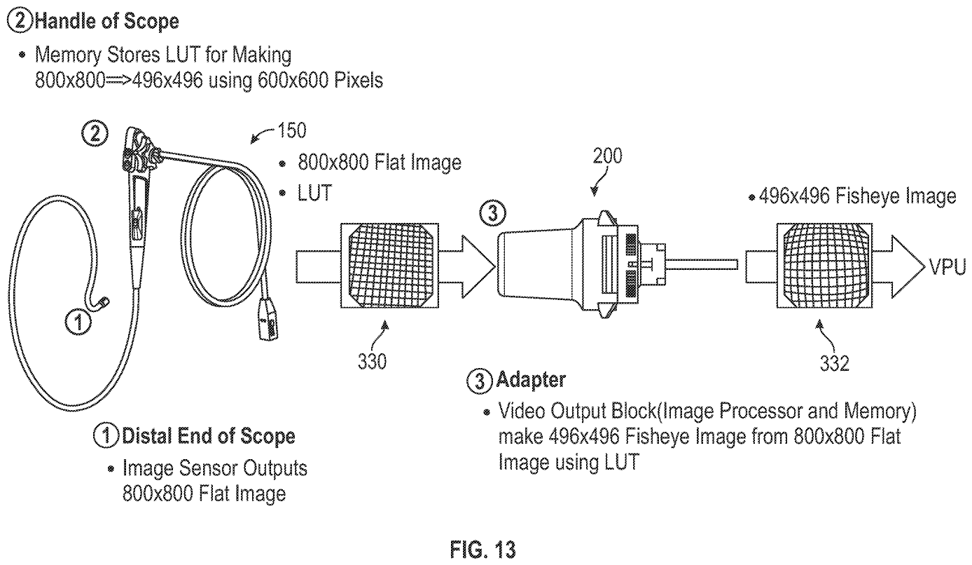

Plug component 204 can be inserted into socket 37 (FIG. 1) of light source unit 22. When inserted, electrical leads 212 and 214 can connect to electrical contacts within socket 37 to allow for transmission of electrical signals from imaging and control system 12, such as from control unit 16 and light source unit 22 to an endoscope. As discussed herein, adapter 200 can include circuitry for performing image processing operations on imaging signals coming into adapter 200 at electrical coupler 232 and convey such processed signals to electrical leads 212. Additionally, adapter 200 can be configured to receive imaging signals via other means, such as via wireless communication signals, such as by using wireless communication device 260 (FIG. 11).

A plug for endoscope 152 (FIG. 4) can be inserted into opening 222. Opening 222 can have an irregular shape, such as a generally square shape with one side being rounded, to facilitate assembly with an endoscope plug in one orientation. Alignment posts 234A and 234B can be located in opening 222 to facilitate coupling with the endoscope plug. For example, alignment posts 234A and 234B can comprise cylindrical posts over which cylindrical sockets in the endoscope plug can slide to facilitate alignment. Additionally, alignment posts 234A and 234B can relive stress from being applied to fluid coupler 230 and electrical coupler 232. In additional examples, alignment posts 234A and 234B can be spring loaded to facilitate ejection of adapter 200. For example, alignment posts 234A and 234B can be biased to an extended position and then compressed when adapter 200 is connected to a control unit. As such, the force of the compressed springs can facilitate ejection of adapter 200 when pulled upon by an operator or user.

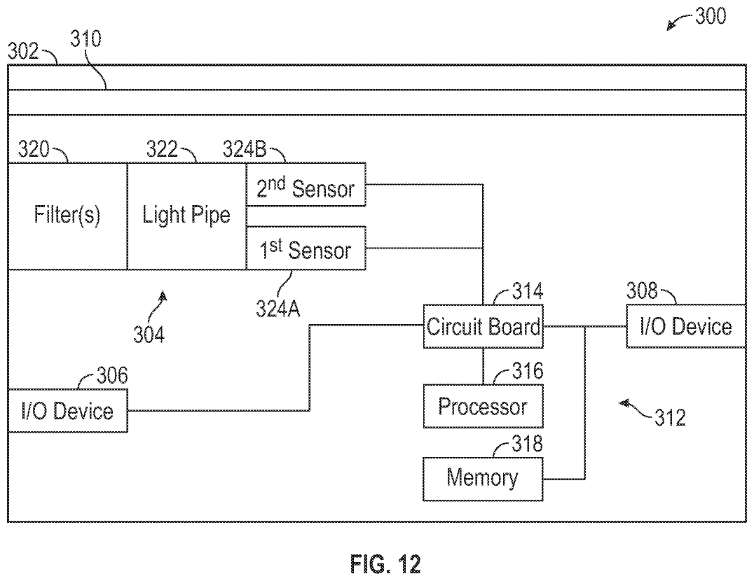

Air lines can be connected to air coupler 208 and air coupler 230. Air line 242 (FIGS. 10 and 11) can extend between air coupler 208 and air coupler 230 to allow air to pass through adapter 200. For example, air coupler 208 and air coupler 230 can be connected to air, carbon dioxide, saline, water and other fluid to perform various functions, including insufflation. In examples, air coupler 208 and air coupler 230 can comprise hose couplers or hose fittings with and without valves. In examples, adapter 200 can be configured to simply allow air to passthrough main housing 202 and plug component 204 without interference, adjustment or control. However, in some examples, adapter 200 can be configured to actively control air flow through main housing 202 and plug component 204 based on received electronic signals from control unit 16 (FIG. 1) or other sources, such as by including electronically controlled valves.

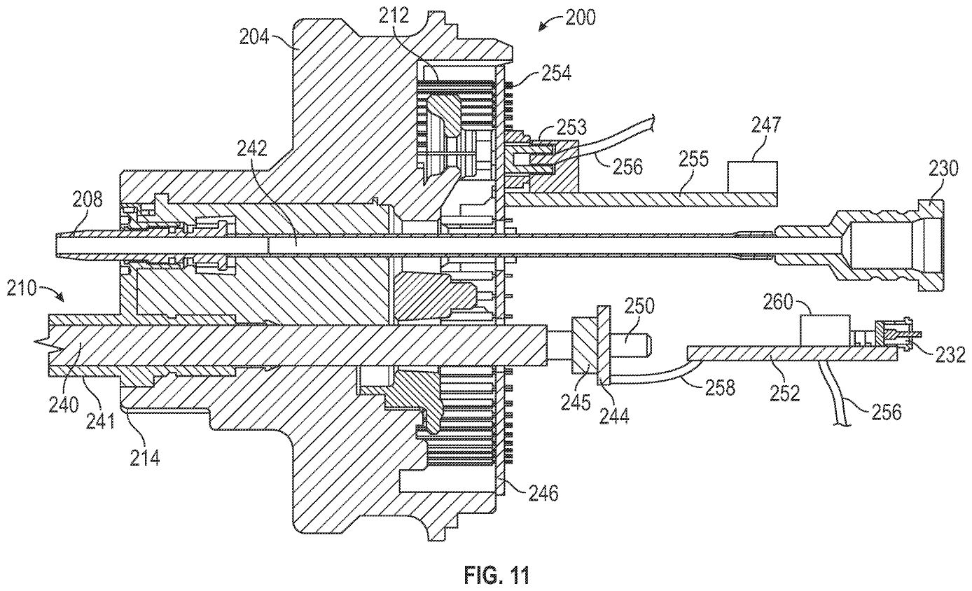

Light conductor assembly 210 can be configured to receive light waves from a light source. In particular, the end of light conductor assembly 210 can face the output of a light bulb or LED within light source unit 22. Light conductor assembly 210 can extend into plug component 204 and discharge the light waves onto sensor package 245 (FIG. 9). Electronics connected to sensor package 245 can translate the light waves into instructions for light generator 166 (FIG. 4) that can be transmitted through electrical coupler 232. In examples, light conductor assembly 210 can comprise light conductor 240 disposed in sheath 241. In examples, light conductor 240 can comprise a light pipe or a bundle of light fibers. For example, light conductor 39 (FIG. 2) of endoscope 14 can comprise a bundle of light fibers because light conductor 39 can comprise the proximal-most end of a bundle of light fibers extending through cable section 34 and insertion section 28. Thus, it is desirable to produce light conductor 39 from a plurality of light fibers to facilitate flexibility. However, light conductor 240 can comprise a light pipe, which can comprise a single piece light conductor having a diameter much larger than individual light fibers. As such, light conductor 240 can be rigid and more robust, such as by being more resistant to heat.

Furthermore, as discussed herein, adapter 200 can include electronics, such as image processing circuitry 247 (FIGS. 9-11), positioned electrically between leads 212 and electrical coupler 232 to perform image processing techniques and transformations to the raw output of an imaging sensor received at electrical coupler 232 to transmit transformed images to imaging and control system 12 (FIG. 1) through electrical leads 212.

FIG. 9 is a front perspective view of adapter 200 of FIGS. 5 and 6 with main housing 202 removed to show support bracket 203 connected to plug component 204 and socket component 206. FIG. 9 additionally shows sensor package 245 and image processing circuitry 247. FIG. 10 is a front perspective view of adapter 200 of FIG. 9 with support bracket 203 and socket component 206 removed to show air coupler 230, electrical coupler 232, light conductor 240 and air line 242. FIGS. 9 and 10 are discussed concurrently.

Light conductor 240 can be connected to light conductor assembly 210 extending from plug component 204. Light conductor can direct light onto sensor package 245. Air line 242 can be connected to air coupler 230 and air coupler 208 (FIGS. 5 and 7). Light board 244 can be connected to control board 246 of plug component 204 via fastener 248 and post 250. Control board 246 can be connected to prongs 254 of electrical leads 212. Control board 246 can be connected to communication board 252 via connector 253, which can be mounted on board 255. Board 255 can additionally include other electronics components for processing imaging signals received from imaging device 160 (FIG. 4). For example, board 255 can be connected to image processing circuitry 247. Board 255 can additionally be connected to one or more buttons, such as button 219 (FIG. 6) accessible from the exterior of main housing 202 to operate various features of adapter 200. Connector 253 can be connected to communication board 252 via wiring 256 (FIG. 11). Light board 244 can be connected to communication board 252 via wiring 258 (FIG. 11). Communication board 252 can be connected to electrical coupler 232 for transmitting control and light generation signals to endoscope 152 (FIG. 4) as well as receiving imaging signals from electrical coupler 232 for transmission to imaging and control system 12 (FIG. 2). As such, when plug component 204 is inserted into socket 37 (FIG. 1), electrical leads 212 can be placed in communication with endoscope 152 through prongs 254, communication board 252 and electrical coupler 232. Likewise, when plug component 204 is inserted into socket 37, air line 242 can be placed in communication with endoscope 152 through air line 242, air coupler 208 and air coupler 230. Additionally, light conductor assembly 210 can be placed in alignment with sensor package 245.

FIG. 11 is a cross-sectional view through adapter 200 of FIG. 10 showing air line 242 through adapter 200 and sensor package 245 positioned proximate light conductor 240.

Air line 242 can comprise a conduit coupled to air coupler 208 and air coupler 230. In examples, air line 242 can comprise a rubber or plastic pipe or tube. Air line 242 can be connected to appropriate fittings on air coupler 208 and air coupler 230 to provide a leak-proof passage through adapter 200. For example, air coupler 208 and air coupler 230 can include barbed fittings over which air line 242 can fit. Air coupler 208 can comprise a male projection that can be fit into a mating female receptacle in socket 37 (FIG. 1). Air coupler 230 can comprise a female receptacle that can receive a mating male projection on a coupler of scope cable 154 (FIG. 4). Air coupler 208 can be rigidly supported by end face 214 of plug component 204 and air coupler 230 can be rigidly supported by socket component 206. Air line 242 can extend unsupported through adapter 200 between air coupler 208 and air coupler 230. As such, support bracket 203 control board 246 can include appropriate openings to allow for the extension of air line 242 therethrough.

Sensor package 245 can comprise one or more light sensors for interpreting various properties of light waves emanating from light conductor 240. Sensor package 245 or other appropriate electronics can convert output of the light sensors into instructions for operating light generator 66 (FIG. 4). The instructions for operating light generator 66 can be communicated to electrical coupler 232, along with other control signals from electrical leads 212. In examples, electrical coupler 232 can comprise an input/output device configured to send and receive electronic communications signals, as well as electrical power, e.g., current. In examples, electrical coupler 232 can comprise a Universal Serial Bus (USB) port, specifically a USB-C port. Thus, output from sensor package 245 and various prongs 254 from control unit 16 can be passed along to various components of endoscope 152 (FIG. 4).

In examples, imaging signals can follow a path including electrical coupler 232, communication board 252, wiring 256, connector 253, board 255 including light processing circuitry 247, control board 246 and electrical leads 212 to reach imaging and control system 12 (FIG. 1). Additionally, in examples, electrical coupler 232 can be replaced or supplemented by wireless communication device 260 to facilitate wireless transmissions to and from communication board 252. Wireless communication device 260 can be wirelessly connected to a complementary wireless communication device of an endoscope to receive digital images. Light processing circuitry 247 can comprise memory, such as random-access memory (RAM), read-only memory (ROM), optical memory, magnetic storage memory, solid-stated drive, and the like. The memory can be used to store the imaging output of imaging sensor 160, as well as lookup tables for processing the imaging output. For example, imaging output can be stored in temporary memory, while lookup tables can be stored in permanent memory. Light process circuitry 247 can include appropriate electronics components, such as circuit boards and processors for transforming the imaging output of imaging sensor 160 using the lookup tables, as is discussed with reference to FIG. 12.

FIG. 12 is a block diagram illustrating light processing adapter 300 of the present disclosure. Light processing adapter 300 can comprise housing 302, light pipe assembly 304, first input/output (I/O) device 306, second input/output (I/O) device 308, air passage 310 and controller 312. Controller 312 can comprise circuit board 314, processor 316 and memory 318. Controller 312 can comprise light processing circuitry 247 of FIGS. 9-11. Light pipe assembly 304 can comprise filters 320, light pipe 322, first sensor 324A and second sensor 324B.

Air passage 310 can be configured similarly as air coupler 208, air line 242 and air coupler 230. Air passage 310 can be configured as a pipe or tube to allow a fluid, such as air, gas, and water, to pass through adapter 300. Ends of air passage 310 can be provide with appropriate male or female fitting to connect to an imaging control system and an endoscope.

I/O device 306 can be configured as, or to communicate with, electrical leads 212, prongs 254 and control board 246. I/O device 306 can be configured to relay electronic communication signals into and out of adapter 300 for communication with an imaging and control system. I/O device 308 can be configured as electrical coupler 232. I/O device 306 can be configured to relay electronic communication signals into and out of adapter 300 for communication with a light-generating endoscope.

In examples, I/O device 306 and I/O device 308 can communicate using wireless communications signals, such as Bluetooth, WiFi, Zigbee, infrared (IR), near field communication (NFC), 3GPP or other technologies. In examples, I/O device 306 and I/O device 308 can comprise wired connections or can include ports for receiving wires for wired connections. In examples, I/O device 306 and I/O device 308 can communicate using one of more of the IEEE 802.15.6-2012 protocol, an MICS protocol and an MBANs protocol. In examples, I/O device 306 and I/O device 308 can comprise a port, such as a serial (e.g., Universal Serial Bus (USB) port, parallel port, or another wired or wireless (e.g., infrared (IR), near field communication (NFC), etc.) connection to communicate or control one or more features of an imaging and control system and endoscope.

Filters 320 can be configured as filters 264A and 264B. In examples, filters 320 can comprise absorptive filters that can absorb wavelengths of certain colors and that allow wavelengths of other colors to pass through. In examples, filters 320 can comprise interference filters that reflect wavelengths in certain spectral bands and that transmits wavelengths in other spectral bands. In examples, filters 320 can comprise a pair of polarizer filters rotationally offset to allows light waves of a specific polarization to pass through while blocking light waves of other polarizations.

Light pipe 322 can be configured as light conductor 240. Light pipe 322 can comprise a single-piece or monolithic component fabricated from optical acrylic or polycarbonate or other materials. In alternative example, light pipe 322 can be replaced by a bundle of optical fibers made of silica or plastic or other materials. Light pipe 322 can extend between filter 320 and sensors 324A and sensor 324B. As such, light exiting filters 320 can enter one end face of light pipe 322 and light exiting the opposite face of light pipe 322 can direct light waves onto sensor 324A and sensor 324B.

First sensor 324A and second sensor 324B can be configured as a portion of sensor package 245 (FIGS. 11 and 12). In examples, first sensor 324A can comprise a light intensity sensor. In examples, first sensor 324A can comprise a photodiode, a photoresistor, a phototransistor, and a photovoltaic light sensor. In examples, second sensor 324B can comprise a color sensor. In examples, second sensor 324B can comprise a light-to-photocurrent conversion sensor, a light-to-analog-voltage conversion sensor, and a light-to-digital conversion sensor.

Circuit board 314 can comprise a structural component for electrically and structurally coupling electrical components of adapter 300. For example, circuit board 314 can comprise a silicon wafer or a chip onto which electrical couplings are attached for electronic coupling of processor 316, memory 318, sensor 324A and sensor 324B and the like. Circuit board 314 as connected to processor 316, memory 318 and sensors 324A and 324B can operate as a converter for converting light waves into electronic signals as described herein.

Processor 316 can comprise an integrated circuit that controls operation of components of adapter 300, such as I/O devices 306 and 308, sensors 324A and 324B and memory 318. Processor 316 can execute instructions stored in memory 318 to operate components of adapter 300, such as sensors 324A and 324B. In examples, a processor and memory are not needed and adapter 300 can operate as a simple integrated circuit whereby output of sensors 324A and 324B can be directly transmitted by I/O devices 306 and 308.

Memory 318 can comprise any suitable storage device, such as non-volatile computer-readable memory, magnetic memory, flash memory, volatile memory, programmable read-only memory and the like. Memory 318 can include instructions stored therein for processor 316 to control operation of adapter 300. In examples, memory 318 can additionally include random access memory (RAM) or other types of memory suitable for storing imaging signals for processing by processor 316 and transmission out of light processing adapter 300 because, for example, long term storage of live-streamed imaging data is typically not needed. Memory 318 can include more stable memory for storing information intended to be repeatedly used by light processing adapter 300 across multiple uses and that can be useful with different endoscopes.

Memory 318 can include instructions for operating I/O devices 306 and 308 and sensors 324A and 324B. Memory 318 can additionally include reference data for comparing to data from sensors 324A and 324B, such as one or more lookup tables (LUTs) for correlating light intensity sensed to a power input to light generator 166 (FIG. 4) and other information, that can be used to convert a light wave of a particular intensity and color into one or more electronic signals for generating light of the same or approximately same intensity and color. In an example, memory 318 can include a lookup table having light intensity from zero to the maximum output of light source 110 (FIG. 4) that is correlated to current input to light generator 166 (FIG. 4) from zero to a maximum input to light generator 166. Memory 318 can additionally include instructions for scaling light signal generated by light generator 16 based on the effects of filters 320, such as by applying scaling factors to adjust the output of light source 110 by a correlated percentage. Memory 318 can additionally include instructions for compensating for degradation in the performance of light source 110 over time, such as by applying a correction factor determined by comparing the actual output of light source 110 to an expected output stored in memory 318.

Memory 318 can additionally include information for processing imaging from imaging sensor 160 (FIG. 4). For example, memory 318 can include one or more lookup tables (LUTs) having imaging processing conversion tables stored therein. The lookup tables can include matrices of pixel locations for flat images from imaging sensor 160 that can be converted into locations in matrices of converted images. The lookup tables can comprise transformation tables that map pixels of a source image to locations in a transformed image. In examples, a lookup table can correlate locations of pixels from a flat image to locations for the pixels in a distorted image. Memory 318 can include lookup tables for shifting or reframing images, such as to remove components of medical devices, e.g., elevators, in the field of view of the imaging device. Memory 318 can include a plurality of tables for different endoscopes and identifiers for different endoscopes that are mapped to different lookup tables in the plurality of lookup tables. Thus, light processing adapter 300 can be used with different endoscopes having different image outputs due to, for example, different types of lenses used therewith. Disposable endoscopes can be configured to output an identification signal that provides the type of imaging sensor, lens or imaging output intended to be received by the imaging and control system. Thus, processor 316 can use information in memory to find the proper lookup table based on the type or model of endoscope to provide the imaging and control system the appropriate imaging signal.

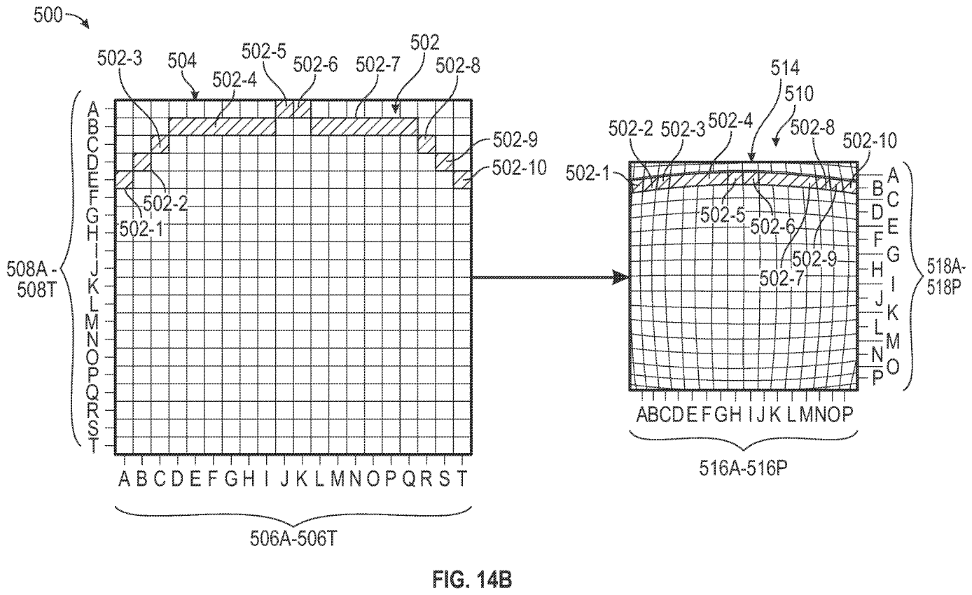

FIG. 13 is a schematic diagram illustrating imaging processing adapter 200 converting flat image 330 obtained from imaging sensor 160 (FIG. 4) to fisheye image 332.

The current reusable duodenoscope has an image sensor and lens designed to produce a fisheye image (greater magnification in the center of target), but this design using an image sensor and customized lens is expensive for a single-use endoscope. The most commonly mass-produced imagers available have lensing that produce a flat image, which would not be as desirable for a physician accustomed to seeing a fisheye image.

Therefore, there is a need to create a fisheye image while utilizing the mass-produced image sensor which will not substantially increase the cost of the endoscope.

In examples, the present disclosure relates to modifying the existing flat image without additional refraction lensing applied to the image sensor, which would add significant cost and space penalties. The flat image is modified electronically using a strategy of hardware and software to produce the desired barrel distortion. The screen image is saved to a RAM chip in the system (preferably in the adapters of the present disclosure), along with a FPGA (field programmable gate array), which then is programmed to manipulate pixels before transmitting to the VPU (video processing unit), such as imaging and control system 12 (FIG. 1) for display.

An image sensor is located at a distal end of endoscope, the image sensor outputs an 800×800 flat image. An adapter which is connected between the endoscope and the VPU (video processor unit) converts the flat image to fisheye image using a video output block stored in a RAM chip and selectively manipulating the image via the FPGA and a LUT (lookup table), which provides the instructions on how the scaling should occur across the screen image. The resultant image is cropped and downsized to produce a 100 deg field of view from the 120 deg generated from the mass-produced imager lensing which is required for the intended design. Alternately, for different applications, the image could minimize cropping and scaling to keep the resultant image as close to the sensor specifications as possible.

While this example is used for implementing barrel distortion, other image manipulations can be performed by saving the image in RAM and altering it using an FPGA. For example, pincushion distortion or image skewing could be implemented, zoom, scaling and cropping could be limited to parts of the image such as the center or user identified portions. By storing images to RAM, stitching of multiple images to create a larger field of view or create higher resolution images may be used with a higher framerate camera system. Additionally storing sequential images could be used to create stereoscopic images for 3D effects.

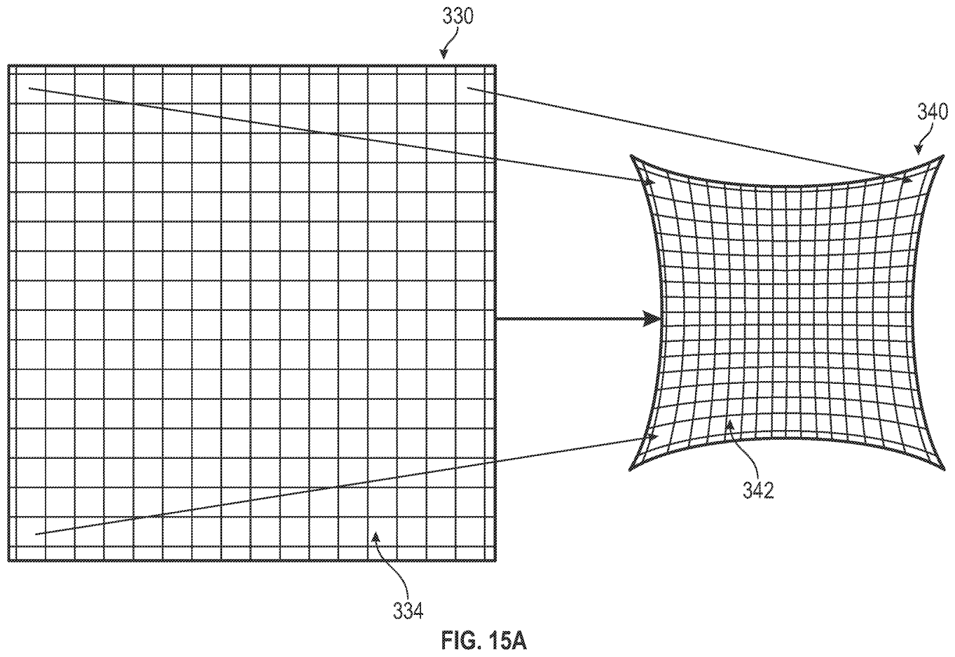

FIG. 14A is a schematic diagram of flat image 330 being transformed to fisheye image 332 via an image manipulation process. Fisheye image 330 can comprise a warped image.

Flat image 330 incoming from imaging sensor 160 (FIG. 4) can comprise a flat image that is sized at 800 pixels×800 pixels. The pixels can be mapped to grid locations for the flat image. For example, x and y coordinates can be assigned to each pixel.

Fisheye image 332 desired to be output at output unit 18 (FIG. 1) can have a fisheye-shaped distortion and can be sized at 496 pixels×496 pixels. As such, the image processing adapters of the present disclosure can perform cropping of flat image 330 to, for example, transform the output of imaging sensor 160 (FIG. 4) to what is similar to imaging sensor 120 (FIG. 3), which can have a different type of imaging sensor.

Fisheye image 332 can include pixels from flat image 330 that have been moved into different locations. Fisheye image 332 can have grid locations having x and y coordinates assigned thereto. However, the x and y coordinates can be spaced further apart toward the center of fisheye image 332 than at the periphery. Additionally, some of the pixels from flat image 330 can be omitted. For example, some of the pixels around the periphery of flat image 330 can be omitted to, for example, accommodate or make space for the expanded pixels around the center. The pixel mapping transformation for flat image 220 to fisheye image 330 can be stored in a lookup table.

Image processing adapter 200 can perform scaling and cropping in one move or conversion operation. Image processing adapter 200 can comprise one or more lookup tables to instruct a video output block, such as image processing circuitry 247 (FIGS. 9-11), where to pull pixels from flat image 330 and where to put the pulled pixels in fisheye image 332. In examples, lookup tables for image transformation can be stored in the handle of endoscope 152 (FIG. 4). As such, each endoscope configured for use with image processing adapter 200 can instruct image processing adapter 200 the desired transformation to make output that is similar to a non-disposable or reusable endoscope.

FIG. 14B is a diagram of digital image 500 having pixels 502 disposed within grid structure 504. FIG. 14B also shows a diagram of transformed digital image 510 wherein pixels 502 from digital image 500 have been distributed into barrel grid structure 514. FIG. 14B illustrates a particular transformation for the image manipulation process described with reference to FIG. 14A.

Digital image 500 can represent the raw output of an image sensor, such as imaging sensor 160 of FIG. 4. Transformed digital image 510 can comprise a version of digital image 500 processed by an image processing device of the present disclosure, such as adapter 150 of FIG. 4. As disclosed herein, transformed digital image 510 can have one or more operations performed thereon, such as cropping, zooming and shaping, to produce a digital image of a desired format, resolution, size and distortion. In the example of FIG. 14B, transformed digital image 510 is cropped to a reduced image size, e.g., an image having fewer pixels, and transformed, e.g., distorted or stretched at a central location into a fisheye shape.

Grid structure 506 can comprise column 506A-column 506T and row 508A-row 508T. Column 506A-column 506T can comprise pixels 502 that are arranged in straight vertical lines, relative to the orientation of FIG. 14B. Row 508A-row 508T can comprise pixels 502 that are arranged in straight horizontal lines, relative to the orientation of FIG. 14B. Each of pixels 502 can be square. As such, digital image 500 can comprise a rectilinear space. FIG. 14B illustrates digital image 500 as being a square with equal quantities of rows and columns. However, digital image 500 can have a rectangular shape such that there are more rows or more columns of pixels 502.

Grid structure 514 can comprise column 516A-column 516P and row 518A-row 518P. Column 516A-column 516P can comprise pixels 502 that are arranged in outwardly curved (e.g., away from the center of grid structure 514) lines, relative to the orientation of FIG. 14B. Row 518A-row 518P can comprise pixels 502 that are arranged in outwardly curved (e.g., away from the center of grid structure 514) lines, relative to the orientation of FIG. 14B. Each of pixels 502 can be a distorted square. In examples, pixels at or near the center of transformed digital image 510 can be stretched larger than pixels at the center of digital image 550. However, pixels at or near the center of transformed digital image 510 can be less distorted or not distorted. The further away from the center of transformed digital image 510, pixels 502 can become more distorted. Thus, pixels 502 near the center of transformed digital image 510 can be square, while pixels 502 toward the four corners of transformed digital image 510 can be shaped like a rhombus, equilateral quadrilateral or diamond, and can also have curved sides. However, other shapes, including other fisheye shapes, can be produced. Transformed digital image 510 can comprise a rectilinear shape having outwardly bowed edges. However, the edges of transformed digital image 510 can be truncated to form straight edges, as illustrated. FIG. 14B illustrates transformed digital image 510 as being a square with equal quantities of rows and columns. However, transformed digital image 510 can have a rectangular shape such that there are more rows or more columns of pixels 502.

Image processing hardware and software of adapter 150 can assign grid coordinates to the columns and rows within digital image 500. Likewise, the columns and rows of transformed digital image 510 can include grid coordinates. Adapter 150 can copy information regarding any or all of pixels 502 in digital image 500, process the pixel information, and then reproduce the processed pixel information into transformed digital image 500. For example, adapter 150 can take any combination of the color, shade, contrast, brightness, light intensity and other information from one or more of pixels 502 in digital image 500 and transfer some or all of that information, in an unaltered or altered state, to a selected location within transformed digital image 510. In examples, not all of pixels 502 of digital image 510 is transferred to transformed digital image 510. As mentioned, adapter 150 can include a memory device, or can be in communication with a memory device via a wired or wireless connection, that includes mapping information for moving pixels from one coordinate system to another to produce one or more fisheye effects.

FIG. 14B shows an example of five rows of digital image 500 being transformed into a single row in transformed digital image 510 near the top edge of digital image 500 to produce a fisheye effect near the top edge of transformed digital image 510. As such, approximately one hundred pixels from the top portion of digital image 500 are reduced down to approximately sixteen pixels for the top line of transformed digital image 510.

Pixel 502-1 from coordinate A, E, e.g., column A, row E, in digital image 500 can be moved to coordinate A, B in transformed digital image 510. Pixels 502 above pixel 502-1, e.g., pixels in coordinates A, D, to A, A, can be omitted from the transfer.

Pixel 502-2 from coordinate B, D in digital image 500 can be moved to coordinate B, B in transformed digital image 510. Pixels 502 above pixel 502-2, e.g., pixels in coordinates B, C, to B, A, can be omitted from the transfer.

Pixel 502-3 from coordinate C, C in digital image 500 can be moved to coordinate C, B in transformed digital image 510. Pixels 502 above pixel 502-3, e.g., pixels in coordinates C, B, to C, A, can be omitted from the transfer.

Pixels 502-4 can comprise a block of pixels 502 in the same row. In the illustrated example, pixels 502-4 can comprise six pixels. Pixels 502-4 can be moved from coordinates D, B to I, B in digital image 500 to coordinates D, B to G, B in transformed digital image 510. Thus, six pixels 502 of digital image 500 can be trimmed down to four pixels in transformed digital image 510. Pixels 502 above pixels 502-4, e.g., pixels in coordinates D, A to coordinates I, A, can be omitted from the transfer.

Pixel 502-5 from coordinate J, A in digital image 500 can be moved to coordinate H, B in transformed digital image 510.

Pixel 505-6 can be moved similarly form digital image 500 to pixel 502-5 on the other side of transformed digital image 510.

Pixels 505-7 can be moved similarly form digital image 500 to pixels 502-4 on the other side of transformed digital image 510.

Pixel 505-8 can be moved similarly form digital image 500 to pixel 502-3 on the other side of transformed digital image 510.

Pixel 505-9 can be moved similarly form digital image 500 to pixel 502-2 on the other side of transformed digital image 510.

Pixel 505-10 can be moved similarly form digital image 500 to pixel 502-1 on the other side of transformed digital image 510.

Thus, the left-hand side of transformed digital image 510 can be a mirror image of the right-hand side of transformed digital image 510 in terms of the mapping process, though the content of each pixel is independent.

FIG. 14B illustrates an example where a vertically spaced pattern of pixels 502 from multiple straight rows in digital image 500 can be moved to form a contiguous pattern of pixels 502 in a single inwardly curved row, thereby producing a fisheye effect.

Pixels 502 near the bottom of digital image 500 can be moved to the bottom of transformed digital image in a mirror imaged pattern as at the top.

Pixels near the left edge of digital image 500 can be transferred similarly as the pixels at the top of digital image 500, but rotated ninety degrees counter-clockwise.

Pixels near the right edge of digital image 500 can be transferred similarly as the pixels at the top of digital image 500, but rotated ninety degrees clockwise.

Pixels 502 near the center of digital image 500 can be transferred to transformed digital image 500 in more of a one-to-one correlation. For example, pixels 502 at coordinates (J, J), (J, K), (K, J) and (K, K) of digital image 500 can be moved directly to coordinates (H, H), (H, I), (I, H) and (I, I) in transformed digital image 510.

As discussed, the particular mapping of coordinates from digital image 500 to coordinates for transformed digital image 510 can be stored in memory of image processing circuitry 247 (FIGS. 9-11).

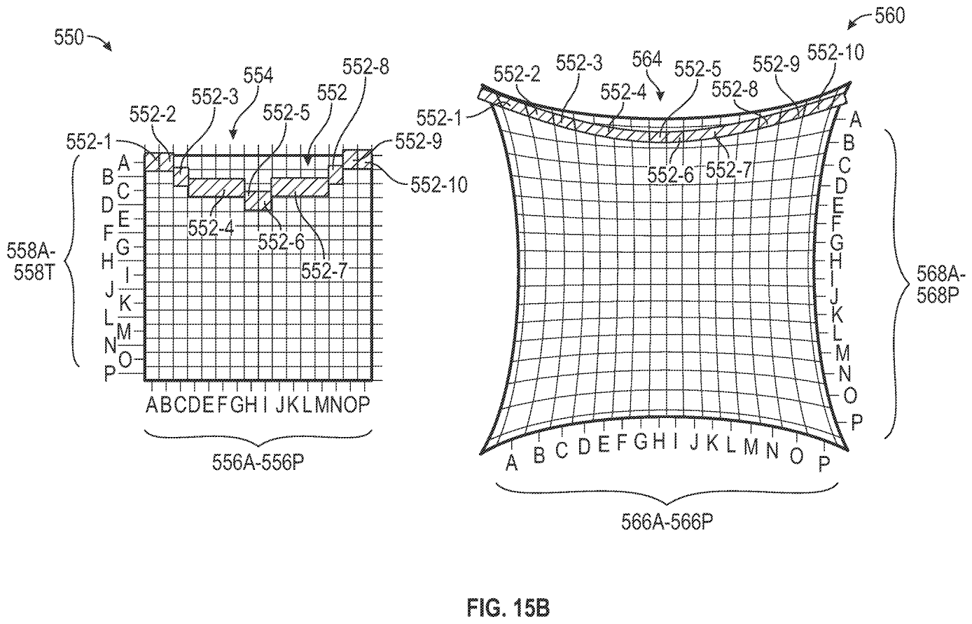

FIG. 15A is a schematic diagram of flat image 330 being transformed to pincushion image 340. Pincushion image 340 can comprise a warped image. Pincushion image 340 can comprise a type of deformation of flat image 330 that is the opposite of fisheye image 330. Pincushion image 340 can be formed in a similar manner as fisheye image 330 of FIG. 14A. Lines of pixels for pincushion image 340 can be pulled from different lines of flat image 330. The x and y coordinates for pincushion image 340 can be spaced closer together toward the center of pincushion image 340 than at the periphery. The pixel mapping transformation for flat image 220 to pincushion image 340 can be stored in a lookup table.

There can be different types of memory used to store information relating to flat image 330, fisheye image 332 and pincushion image 340. Block memory can be used for warping transformations. Data for the source flat image 330 can be streamed to DDR memory. In examples, approximately 70 lines of source image can be loaded into block memory. Video output block can utilize lookup tables to determine the location of needed pixels in source image. Final pixel is streamed to image and control system 12 (FIG. 1).

In additional examples, pixel interpolation can be performed. Interpolation can involve combining pixels of flat image 330 into a single pixel for fisheye image 332 or pincushion image 340 or separating a single pixel for fisheye image 332 into multiple pixels for fisheye image 332 or pincushion image 340. Nearest neighbor interpolation can be used. Additionally Bilinear and Bicubic interpolation can be used. Special filtering can be performed simultaneously with interpolation, if desired. For example, color filtering can be performed to enhance, filter out or add in desirable colors. Other filters can include NBI (Narrow Band Imaging), UV, color enhancement, infrared or shift in color (warm-cool or cool-warm). Besides color filtering, other filters can be performed such as edge enhancement (sharpness).

FIG. 15B is a diagram of digital image 550 having pixels 552 disposed within grid structure 554. FIG. 14B also shows a diagram of transformed digital image 560 wherein pixels 552 from digital image 550 have been distributed into barrel grid structure 564. FIG. 15B illustrates a particular transformation for the image manipulation process described with reference to FIG. 15A.

Digital image 550 can represent the raw output of an image sensor, such as imaging sensor 160 of FIG. 4. Transformed digital image 560 can comprise a version of digital image 550 processed by an image processing device of the present disclosure, such as adapter 150 of FIG. 4. As disclosed herein, transformed digital image 560 can have one or more operations performed thereon, such as cropping, zooming and shaping, to produce a digital image of a desired format, resolution, size and distortion. In the example of FIG. 15B, transformed digital image 560 is transformed, e.g., distorted or stretched at a central location into a pincushion shape. In the illustrated example, transformed digital image 510 is not cropped to a reduced image size, e.g., an image having fewer pixels, but can be in other pincushion transformations.

Grid structure 554 can comprise column 556A-column 556P and row 558A-row 558P. Column 556A-column 556P can comprise pixels 552 that are arranged in straight vertical lines, relative to the orientation of FIG. 15B. Row 558A-row 558P can comprise pixels 552 that are arranged in straight horizontal lines, relative to the orientation of FIG. 15B. Each of pixels 552 can be square. As such, digital image 550 can comprise a rectilinear space. FIG. 15B illustrates digital image 550 as being a square with equal quantities of rows and columns. However, digital image 550 can have a rectangular shape such that there are more rows or more columns of pixels 552.

Grid structure 564 can comprise column 566A-column 566P and row 568A-row 568P. Column 566A-column 566P can comprise pixels 552 that are arranged in inwardly curved (e.g., toward the center of grid structure 554) lines, relative to the orientation of FIG. 15B. Row 568A-row 568P can comprise pixels 552 that are arranged in inwardly curved (e.g., toward the center of grid structure 554) lines, relative to the orientation of FIG. 15B. Each of pixels 552 can be a distorted square. However, pixels at or near the center of transformed digital image 560 can be less distorted or not distorted. In examples, pixels at or near the periphery of transformed digital image 560 can be stretched larger than pixels at the periphery of digital image 550. The further away from the center of transformed digital image 560, pixels 552 can become more distorted. Thus, pixels 552 near the center of transformed digital image 560 can be square, while pixels 552 toward the four corners of transformed digital image 560 can be shaped like a rhombus, equilateral quadrilateral or diamond, and can also have curved sides. However, other shapes, including other pincushion shapes, can be produced. Transformed digital image 560 can comprise a rectilinear shape having inwardly bowed edges, as illustrated. However, the edges of transformed digital image 560 can be truncated to form straight edges. FIG. 15B illustrates transformed digital image 560 as being a stretched square with equal quantities of rows and columns. However, transformed digital image 560 can have a rectangular shape such that there are more rows or more columns of pixels 552.

Image processing hardware and software of adapter 160 can assign grid coordinates to the columns and rows within digital image 550. Likewise, the columns and rows of transformed digital image 560 can include grid coordinates. Adapter 160 can copy information regarding any or all of pixels 552 in digital image 550, process the pixel information, and then reproduce the processed pixel information into transformed digital image 550. For example, adapter 150 can take any combination of the color, shade, contrast, brightness, light intensity and other information from one or more of pixels 552 in digital image 550 and transfer some or all of that information, in an unaltered or altered state, to a selected location within transformed digital image 560. In examples, not all of pixels 552 of digital image 560 is transferred to transformed digital image 560. As mentioned, adapter 150 can include a memory device, or can be in communication with a memory device via a wired or wireless connection, that includes mapping information for moving pixels from one coordinate system to another to produce one or more pincushion effects.

FIG. 15B shows an example of four rows of digital image 550 being transformed into a single row in transformed digital image 560 near the top edge of digital image 550 to produce a pincushion effect near the top edge of transformed digital image 560. As such, approximately sixty-four pixels from digital image 550 are reduced down to approximately sixteen pixels in transformed digital image 560.

Pixel 552-1 from coordinate A, A, e.g., column A, row A, in digital image 550 can be moved to coordinate A, A in transformed digital image 560.

Pixel 552-2 from coordinate B, A in digital image 550 can be moved to coordinate B, A in transformed digital image 560.

Pixel 552-3 from coordinate C, B in digital image 550 can be moved to coordinate C, A in transformed digital image 560. Pixels 552 above pixel 552-3, e.g., the pixel in coordinates C, A, can be omitted from the transfer.

Pixels 552-4 can comprise a block of pixels 552 in the same row. In the illustrated example, pixels 552-4 can comprise four pixels. Pixels 552-4 can be moved from coordinates D, C to G, C in digital image 550 to coordinates D, A to G, A in transformed digital image 560. Thus, four pixels 552 of digital image 550 can be transferred to four pixels in transformed digital image 560. Pixels 552 above pixels 552-4, e.g., pixels in coordinates D, B to G, B and coordinates D, A to G, A, can be omitted from the transfer.

Pixel 552-5 from coordinate H, D in digital image 550 can be moved to coordinate H, A in transformed digital image 560. Pixels 552 above pixel 552-5, e.g., pixels in coordinates H, C, to H, A, can be omitted from the transfer.

Pixel 552-6 can be moved similarly form digital image 550 to pixel 552-5 on the other side of transformed digital image 560.

Pixels 552-7 can be moved similarly form digital image 550 to pixels 552-4 on the other side of transformed digital image 560.

Pixel 552-8 can be moved similarly form digital image 550 to pixel 552-3 on the other side of transformed digital image 560.

Pixel 552-9 can be moved similarly form digital image 550 to pixel 552-2 on the other side of transformed digital image 560.

Pixel 552-10 can be moved similarly form digital image 550 to pixel 552-1 on the other side of transformed digital image 560.

Thus, the left-hand side of transformed digital image 560 can be a mirror image of the right-hand side of transformed digital image 560 in terms of the mapping process, though the content of each pixel is independent.

FIG. 15B illustrates an example where a vertically spaced pattern of pixels 552 from multiple straight rows in digital image 550 can be moved to form a contiguous pattern of pixels 552 in a single outwardly curved row, thereby producing a pincushion effect.

Pixels 552 near the bottom of digital image 550 can be moved to the bottom of transformed digital image in a mirror imaged pattern as at the top.

Pixels near the left edge of digital image 550 can be transferred similarly as the pixels at the top of digital image 550, but rotated ninety degrees counter-clockwise.

Pixels near the right edge of digital image 550 can be transferred similarly as the pixels at the top of digital image 550, but rotated ninety degrees clockwise.

Pixels 552 near the center of digital image 550 can be transferred to transformed digital image 550 in more of a one-to-one correlation. For example, pixels 552 at coordinates (H, H), (H, I), (I, H) and (I, I) of digital image 550 can be moved directly to coordinates (H, H), (H, I), (I, H) and (I, I) in transformed digital image 560.

As discussed, the particular mapping of coordinates from digital image 550 to coordinates for transformed digital image 560 can be stored in memory of image processing circuitry 247 (FIGS. 9-11).

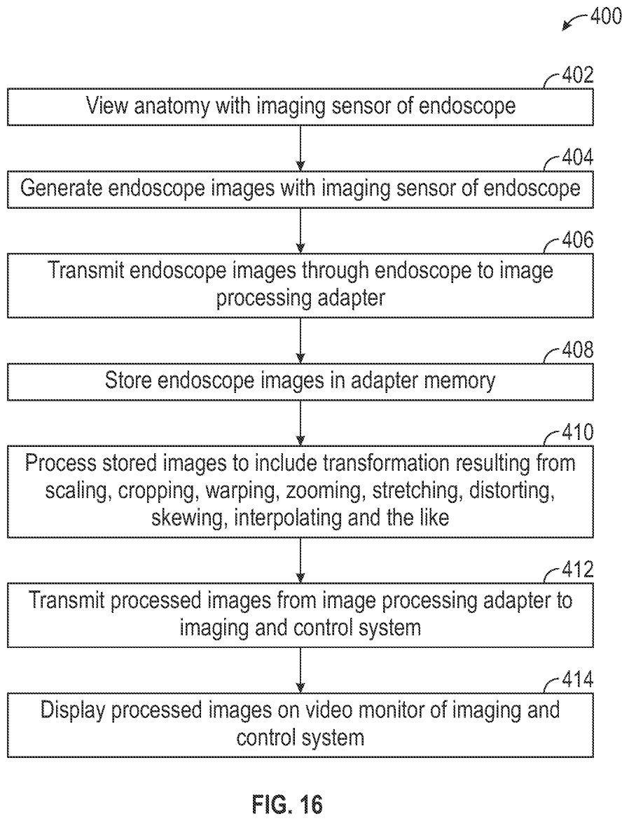

FIG. 16 is a block diagram illustrating operations of method 400 for converting an imaging signal generated by endoscope 152 into a processed imaging signal for imaging and control system 12.

At operation 402, anatomy of a patient can be imaged with an imaging sensor of an endoscope inserted into the patient. Light reflected from the anatomy can enter imaging device 160 (FIG. 4). In examples, light can pass through lens 162. As discussed, lenses of disposable endoscopes can be configured to not bend the light. As such, lens 162 can be omitted.

At operation 404, imaging device 160 can convert the light into an imaging signal or video feed. The imaging signal can output a flat image having a fixed field of view and pixel size. For example, a 120° field of view can generate an 800 pixel×800 pixel image.

At operation 406, the imaging signal can be conveyed through endoscope 152. For example, the imaging signal can be conducted through signal wiring 170 to adapter 150.

At operation 408, adapter 150 can store the imaging signal on memory. Multiple lines of source imaging signal can be stored in memory. Storage of the imaging signal in memory can facilitate on-board imaging processing at adapter 150. The imaging signal can be temporarily stored so that adapter 150 can be used with other endoscopes in other procedures without needing to be cleared of data that is no longer of interest.

At operation 410, adapter 150 can perform imaging signal transformation or processing on the stored imaging signal. Processing of the stored imaging signal can comprise zooming, cropping, stretching, warping, distorting, scaling, skewing, interpolating and other image processing techniques. Processing of the stored imaging signal can comprise transforming a matrix of pixels of an image from imaging sensor 160 to a matrix of pixels of a transformed image. The transformation can comprise referencing one or more lookup tables to transform the pixels. The transformation can comprise converting a flat image to a warped image, such as a fisheye image or a pincushion image. The transformation can involve cropping such as reducing the 800×800 image to a 496×496 image. In examples, processing of the stored imaging signal can occur automatically. In examples, adapter 150 of the present disclosure can be configured to perform particular pre-set image processing. Thus, a user can select an adapter 150 to perform desired image processing. In such examples, the image processing can occur automatically in the background without any user intervention. Thus, the image processing can appear invisible to the user. In examples, adapter 150 can be configured to perform all of the processing described herein, but certain processing can be disabled during manufacturing or after manufacturing such that the adapter can perform a more limited subset of the processing described herein. In additional examples, processing of the stored imaging signal can occur based on user inputs. Adapter 150 can include buttons or switches to allow various processing features to be activated or deactivated. Thus, the image processing can be user-initiated and can be turned on or off before or during a medical procedure. A user can therefore toggle between different processed images to obtain the best view or a preferred view. In additional examples, adapter 150 can be programmed when connected to imaging and control system 12. For example, imaging and control system 12 can recognize when adapter 150 is connected to socket 37 (FIG. 1) and can automatically bring up a menu of options on output unit 18. Imaging and control system 12 can additionally recognize a type of endoscope connected to adapter 150.

Output Unit 18 can display image processing capabilities of adapter 150, such as zoom, crop, stretch, warp, distort, scale, skew, interpolate and other image processing, and provide for a user option to activate or deactivate each image processing capability taking into account hardware of the attached endoscope. A user can utilize input unit 20 (FIG. 1) to select active image processing capabilities for adapter 150.

At operation 412, the processed imaging signal can be transmitted to imaging and control system 12 (FIG. 1).

At operation 414, processed imaging signals can be displayed on output unit 18 (FIG. 1).

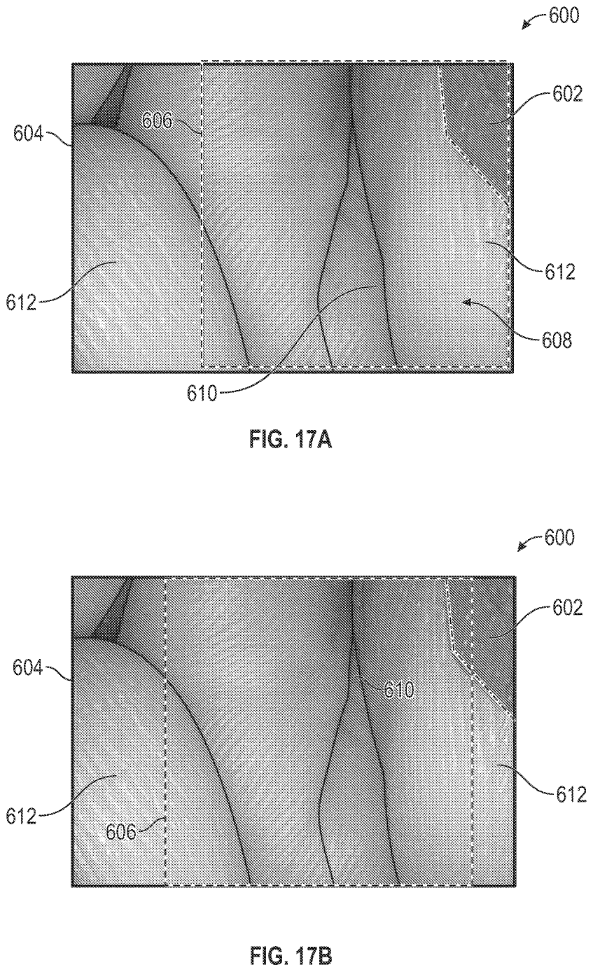

FIG. 17A is a diagram illustrating digital image 600 from an imaging sensor showing the presence of a medical instrument component 602 in field of view 604 and processed image boundary 606 on digital image 600. Digital image 600 can comprise the raw output of an imaging device, such as an imaging device onboard an endoscope. Digital image 600 can represent the entire output of the imaging sensor, e.g., the full resolution. In examples, digital image 600 can comprise 800 pixels×496 pixels. Processed image boundary 606 can comprise a portion of digital image 600 that is to be utilized for transmission to control unit 16 and displayed on output unit 18 of FIGS. 1 and 2. Processed image boundary 606 can comprise 496 pixels×496 pixels. Thus, processed image boundary 606 can be cropped relative to digital image 600. Processed image boundary 606 can additionally be transformed into a pincushion or fisheye shape as described herein.

Digital image 600 can be generated while inside anatomy of a patient. As such, tissue 608 can be present within digital image 600. Tissue 608 can include feature of interest 610 and surrounding anatomy 612. Digital image 600 can also show medical instrument component 602 in field of view 604 proximate feature of interest 610. In examples, medical instrument component 602 can comprise a portion of an elevator of a duodenoscope. Sometimes it can be desirable to visualize the placement of feature of interest 610 with surrounding anatomy 612 unobstructed to, for example, obtain better context for feature of interest 610 or to better diagnose the condition of feature of interest 610 and surrounding anatomy 612. Thus, the presence of medical instrument component 602 within processed image boundary 606 can obscure a portion of tissue 608 that could be used to evaluate feature of interest 610 and surrounding anatomy 612.

FIG. 17B is a diagram illustrating digital image 600 of FIG. 17B with processed image boundary 606 shifted to adjust the presence and/or location of medical instrument component 602. As discussed herein, processed image boundary 606 can be cropped to, for example, reduce the resolution of processed image boundary 606 to that of a conventional endoscope resolution. Furthermore, processed image boundary 606 can be shifted to re-frame the content of processed image boundary 606. For example, processed image boundary 606 can be shifted to position a greater amount of medical instrument component 602 visible within digital image 600 out of processed image boundary 606. In examples, processed image boundary 606 can be positioned so that medical instrument component 602 is completely out of processed image boundary 606. However, it can be desirable to have a small portion of medical instrument component 602 within processed image boundary 606 for reference and orientation purposes. For example, the presence of medical instrument component 602 can inform a surgeon or anyone viewing digital image 600 the direction in which an auxiliary scope, such as a cholangioscope, is facing relative to a duodenoscope. In examples, processed image boundary 606 can be shifted to position feature of interest 610 more prominently within processed image boundary 606, such as closer to the center. In examples, processed image boundary 606 can be shifted to position more of, e.g., a greater surface area of, feature of interest 610 within processed image boundary 606, such as closer to the center.

Thus, the image processing adapters of the present disclosure can crop raw digital images to reproduce digital images that are obtained from conventional endoscope image processors. Thus, the size of the image can be familiar to the surgeon, for example. In view of the foregoing, the image processing adapters of the present disclosure need not simply choose the center of the raw video image to be passed along so that equal portions of the boundary of the raw video image are cropped out. The image processing adapters can select portions of the raw video image to pass along to the display monitor at the imaging and control system. In examples, image processing circuitry 247 (FIGS. 9-11) can include or be connected to memory having instructions stored therein for executing commands to locate the presence of objects within digital image 600. For example, image processing circuitry can include a database of digital files of the shape and size of various medical instrument components, such as elevators that can be identified by comparison of shapes from video images. Additionally, image processing circuitry 247 can be trained to look for the presence of straight lines within digital image 600. Once image processing circuitry 247 locates a predetermined shape or the presence of straight lines, image processing circuitry 247 can shift the location of processed image boundary 606 to minimize or eliminate the presence of such features within processed image boundary 606 according to programmed parameters. For example, the identified presence of an elevator can trigger shifting the location of the elevator within processed image boundary 660 so that only a predetermined portion or length of the elevator is visible. In examples, image processing circuitry 247 can include or can be in communication with artificial intelligence or machine learning engines that are trained to identify the presence of features to be moved out of processed image boundary 660 or moved more prominently into the middle of processed image boundary 660. In additional examples, a user can utilize buttons on one or any of adapter 150, output unit 18, and input unit 20 to provide manual shifting of processed image boundary 606 relative to digital image 600.

As discussed herein, the present disclosure is useful in receiving imaging signals from disposable endoscopes, or reusable endoscopes, having on-board imaging sensors, which can comprise inexpensive imaging sensors that provide flat imaging signals of a fixed resolution. The image processing adapters of the present disclosure can receive imaging signals from on-board imaging sensors of disposable endoscopes of a fixed and flat output and transform such fixed and flat imaging signals to processed imaging signals that can replicate shaped, e.g., stretched, zoomed, scaled, cropped, and warped imaging signals that replicate output of more expensive, more sophisticated imaging sensors of reusable endoscopes. The image processing adapters of the present disclosure can automatically perform the processing procedures described herein so that such image processing is invisible to the user. However, in other examples, the image processing procedures described herein can be selected by a user, adjusted by the user, or turned off and on by the user. As such, disposable endoscopes having simple imaging sensors can be used with existing capital equipment, such as imaging and control systems, while additionally producing high quality images in formats that surgeons are familiar with from conventional, reusable endoscopes.

### Notes

The above detailed description includes references to the accompanying drawings, which form a part of the detailed description. The drawings show, by way of illustration, specific embodiments in which the invention can be practiced. These embodiments are also referred to herein as “examples.” Such examples can include elements in addition to those shown or described. However, the present inventor also contemplates examples in which only those elements shown or described are provided. Moreover, the present inventor also contemplates examples using any combination or permutation of those elements shown or described (or one or more aspects thereof), either with respect to a particular example (or one or more aspects thereof), or with respect to other examples (or one or more aspects thereof) shown or described herein.

In the event of inconsistent usages between this document and any documents so incorporated by reference, the usage in this document controls.

In this document, the terms “a” or “an” are used, as is common in patent documents, to include one or more than one, independent of any other instances or usages of “at least one” or “one or more.” In this document, the term “or” is used to refer to a nonexclusive or, such that “A or B” includes “A but not B,” “B but not A,” and “A and B,” unless otherwise indicated. In this document, the terms “including” and “in which” are used as the plain-English equivalents of the respective terms “comprising” and “wherein.” Also, in the following claims, the terms “including” and “comprising” are open-ended, that is, a system, device, article, composition, formulation, or process that includes elements in addition to those listed after such a term in a claim are still deemed to fall within the scope of that claim. Moreover, in the following claims, the terms “first,” “second,” and “third,” etc. are used merely as labels, and are not intended to impose numerical requirements on their objects.

Method examples described herein can be machine or computer-implemented at least in part. Some examples can include a computer-readable medium or machine-readable medium encoded with instructions operable to configure an electronic device to perform methods as described in the above examples. An implementation of such methods can include code, such as microcode, assembly language code, a higher-level language code, or the like. Such code can include computer readable instructions for performing various methods. The code may form portions of computer program products. Further, in an example, the code can be tangibly stored on one or more volatile, non-transitory, or non-volatile tangible computer-readable media, such as during execution or at other times. Examples of these tangible computer-readable media can include, but are not limited to, hard disks, removable magnetic disks, removable optical disks (e.g., compact disks and digital video disks), magnetic cassettes, memory cards or sticks, random access memories (RAMs), read only memories (ROMs), and the like.

The above description is intended to be illustrative, and not restrictive. For example, the above-described examples (or one or more aspects thereof) may be used in combination with each other. Other embodiments can be used, such as by one of ordinary skill in the art upon reviewing the above description. The Abstract is provided to allow the reader to quickly ascertain the nature of the technical disclosure. It is submitted with the understanding that it will not be used to interpret or limit the scope or meaning of the claims. Also, in the above Detailed Description, various features may be grouped together to streamline the disclosure. This should not be interpreted as intending that an unclaimed disclosed feature is essential to any claim. Rather, inventive subject matter may lie in less than all features of a particular disclosed embodiment. Thus, the following claims are hereby incorporated into the Detailed Description as examples or embodiments, with each claim standing on its own as a separate embodiment, and it is contemplated that such embodiments can be combined with each other in various combinations or permutations. The scope of the invention should be determined with reference to the appended claims, along with the full scope of equivalents to which such claims are entitled.

### EXAMPLES

Example 1 is an adapter for an endoscope system, the adapter comprising: a housing; an input device for receiving an imaging signal input from an endoscope; an imaging signal processor configured to receive the imaging signal input and perform an image processing technique to the imaging signal input to generate an imaging signal output, the imaging signal processing technique configured to alter a display characteristic of the imaging signal input; an output coupler for providing the imaging signal output to an imaging and control system.

In Example 2, the subject matter of Example 1 optionally includes a light conducting element extending into the housing; and a fluid passage extending through the housing, the fluid passage having an inlet and an outlet accessible from the housing.

In Example 3, the subject matter of any one or more of Examples 1-2 optionally include wherein: the input device comprises a USB port; and the output coupler comprises a plurality of electrical leads.

In Example 4, the subject matter of any one or more of Examples 1-3 optionally include wherein the input device comprises a wireless communication device.

In Example 5, the subject matter of any one or more of Examples 1-4 optionally include wherein the imaging signal processor comprises: a processor; and a non-transitory computer readable storage medium having instructions stored therein for performing the imaging processing technique with the processor.

In Example 6, the subject matter of Example 5 optionally includes wherein the imaging processing technique comprises a distortion process.

In Example 7, the subject matter of any one or more of Examples 5-6 optionally include wherein the imaging processing technique comprises transforming a flat image to a barrel image.

In Example 8, the subject matter of any one or more of Examples 5-7 optionally include wherein the imaging processing technique comprises transforming a source pixelated image to a transformed pixelated image using one or more of the following functions: zooming, cropping, stretching, warping, distorting, scaling, skewing and interpolating.

In Example 9, the subject matter of any one or more of Examples 5-8 optionally include wherein the imaging processing technique comprises cropping a source image to shift a center of the source image and move a portion of a medical instrument within the source image toward a boundary of a cropped image.

In Example 10, the subject matter of any one or more of Examples 5-9 optionally include a lookup table having a transformation table for performing the image processing technique, wherein the transformation table maps pixels of a source image to locations in a transformed image.

In Example 11, the subject matter of Example 10 optionally includes wherein the non-transitory computer readable storage medium of the imaging signal processor of the adapter comprises a plurality of lookup tables including the lookup table.

In Example 12, the subject matter of Example 11 optionally includes wherein the non-transitory computer readable storage medium of the imaging signal processor of the adapter further comprises a listing of identifiers for different endoscopes that are mapped to different lookup tables in the plurality of lookup tables.

In Example 13, the subject matter of any one or more of Examples 10-12 optionally include an endoscope connectable to the input device; and a non-transitory computer readable storage medium disposed in the endoscope, the non-transitory computer readable storage medium having stored therein the lookup table.

Example 14 is a surgical endoscope system comprising: an imaging and control system comprising a socket; an endoscope comprising: a shaft comprising an imaging device at a distal end portion; a working channel extending at least partially through the shaft; and an imaging sensor at the distal end portion, the imaging sensor configured to generate an endoscope imaging signal including a flat image; a communication device for transmitting output of the imaging sensor; and an adapter configured to be connected to the socket, the adapter comprising: an input device configured to communicate with the communication device of the endoscope to receive the endoscope imaging signal; a converter configured to transform the flat image of the endoscope imaging signal into a transformed image of an output imaging signal; and an output coupler configured to connect to the socket of the imaging and control system to convey the output imaging signal.

In Example 15, the subject matter of Example 14 optionally includes wherein the converter comprises: a memory device comprising a lookup table correlating locations of pixels from the flat image to locations for the pixels in the transformed image; and a processor configured to receive the endoscope imaging signal and generate the output imaging signal using the lookup table.

In Example 16, the subject matter of Example 15 optionally includes wherein the memory device further comprises: a plurality of lookup tables for different endoscopes; and identifiers for different endoscopes that are mapped to different lookup tables in the plurality of lookup tables.

In Example 17, the subject matter of any one or more of Examples 14-16 optionally include wherein the endoscope comprises: a memory device comprising a lookup table correlating locations of pixels from the flat image to locations for the pixels in the distorted image.

In Example 18, the subject matter of any one or more of Examples 14-17 optionally include wherein: the endoscope comprises a lens for the imaging device that does not distort light entering the imaging device; and the transformed image comprises a fisheye image configured to replicate an image from a fisheye lens positioned over the imaging device.

Example 19 is a method for transforming a flat image to a distorted image, the method comprising: mapping pixels of the flat image to a first table correlating the pixels to locations in the flat image; correlating the locations of the first table to locations in a second table for the distorted image; and generating the distorted image by moving pixels form the locations of the first table to associated locations in the second table.

In Example 20, the subject matter of Example 19 optionally includes receiving the flat image from an endoscope at an adapter; and transmitting the distorted image to an imaging and control system for the endoscope.

In Example 21, the subject matter of Example 20 optionally includes wherein the distorted image simulates a fisheye image obtained from an endoscope having a fisheye lens configured to bend light entering an imaging sensor.

In Example 22, the subject matter of any one or more of Examples 19-21 optionally include wherein generating the distorted image comprises: cropping pixels from the flat image to reduce a size of the flat image; and warping pixels in a central portion of the flat image to generate a fisheye effect.

In Example 23, the subject matter of any one or more of Examples 19-22 optionally include wherein generating the distorted image comprises: performing one or more of the following functions to generate the distorted image from the flat image: zooming, cropping, stretching, warping, distorting, scaling, skewing and interpolating.

In Example 24, the subject matter of any one or more of Examples 19-23 optionally include wherein generating the distorted image comprises: asymmetrically cropping pixels from the flat image relative to a center of the flat image to reposition a portion of a medical instrument visible in the flat image.

1. An adapter for an endoscope system, the adapter comprising:
a housing;
an input device for receiving an imaging signal input from an endoscope;
an imaging signal processor configured to receive the imaging signal input and perform an image processing technique to the imaging signal input to generate an imaging signal output, the image processing technique configured to alter a display characteristic of the imaging signal input; and
an output coupler for providing the imaging signal output to an imaging and control system.

2. The adapter of claim 1, further comprising:
a light conducting element extending into the housing; and
a fluid passage extending through the housing, the fluid passage having an inlet and an outlet accessible from the housing.

3. The adapter of claim 1, wherein:
the input device comprises a USB port; and
the output coupler comprises a plurality of electrical leads.

4. The adapter of claim 1, wherein the input device comprises a wireless communication device.

5. The adapter of claim 1, wherein the imaging signal processor comprises:
a processor; and
a non-transitory computer readable storage medium having instructions stored therein for performing the image processing technique with the processor.

6. The adapter of claim 5, wherein the image processing technique comprises a distortion process.

7. The adapter of claim 5, wherein the image processing technique comprises transforming a flat image to a barrel image.

8. The adapter of claim 5, wherein the image processing technique comprises transforming a source pixelated image to a transformed pixelated image using one or more of the following functions: zooming, cropping, stretching, warping, distorting, scaling, skewing and interpolating.

9. The adapter of claim 5, wherein the image processing technique comprises cropping a source image to shift a center of the source image and move a portion of a medical instrument within the source image toward a boundary of a cropped image.

10. The adapter of claim 5, wherein the non-transitory computer readable storage medium of the imaging signal processor of the adapter comprises a plurality of lookup tables including a lookup table, wherein the lookup table comprises transformation table for performing the image processing technique, wherein the transformation table maps pixels of a source image to locations in a transformed image, wherein the non-transitory computer readable storage medium of the imaging signal processor of the adapter further comprises a listing of identifiers for different endoscopes that are mapped to different lookup tables in the plurality of lookup tables.

11. (canceled)

12. (canceled)

13. The adapter of claim 10, further comprising:
an endoscope connectable to the input device; and
a non-transitory computer readable storage medium disposed in the endoscope, the non-transitory computer readable storage medium having stored therein the lookup table.

14. A surgical endoscope system comprising:
an imaging and control system comprising a socket;
an endoscope comprising:
a shaft comprising an imaging device at a distal end portion;
a working channel extending at least partially through the shaft;
an imaging sensor at the distal end portion, the imaging sensor configured to generate an endoscope imaging signal including a flat image; and
a communication device for transmitting output of the imaging sensor; and
an adapter configured to be connected to the socket, the adapter comprising:
an input device configured to communicate with the communication device of the endoscope to receive the endoscope imaging signal;
a converter configured to transform the flat image of the endoscope imaging signal into a transformed image of an output imaging signal; and
an output coupler configured to connect to the socket of the imaging and control system to convey the output imaging signal.

15. The surgical endoscope system of claim 14, wherein:
the converter comprises:
a memory device comprising a lookup table correlating locations of pixels from the flat image to locations for the pixels in the transformed image; and
a processor configured to receive the endoscope imaging signal and generate the output imaging signal using the lookup table;
wherein the memory device further comprises:
a plurality of lookup tables for different endoscopes; and
identifiers for different endoscopes that are mapped to different lookup tables in the plurality of lookup tables.

16. (canceled)

17. The surgical endoscope system of claim 14, wherein the endoscope comprises:
a memory device comprising a lookup table correlating locations of pixels from the flat image to locations for the pixels in the transformed image.

18. The surgical endoscope system of claim 14, wherein:
the endoscope comprises a lens for the imaging device that does not distort light entering the imaging device; and
the transformed image comprises a fisheye image configured to replicate an image from a fisheye lens positioned over the imaging device.

19. A method for transforming a flat image to a distorted image, the method comprising:
mapping pixels of the flat image to a first table correlating the pixels to locations in the flat image;
correlating the locations of the first table to locations in a second table for the distorted image; and
generating the distorted image by moving pixels from the locations of the first table to associated locations in the second table.

20. The method of claim 19, further comprising:
receiving the flat image from an endoscope at an adapter; and
transmitting the distorted image to an imaging and control system for the endoscope;
wherein the distorted image simulates a fisheye image obtained from an endoscope having a fisheye lens configured to bend light entering an imaging sensor.

21. (canceled)

22. The method of claim 19, wherein generating the distorted image comprises:
cropping pixels from the flat image to reduce a size of the flat image; and
warping pixels in a central portion of the flat image to generate a fisheye effect.

23. The method of claim 19, wherein generating the distorted image comprises:
performing one or more of the following functions to generate the distorted image from the flat image: zooming, cropping, stretching, warping, distorting, scaling, skewing and interpolating.

24. The method of claim 19, wherein generating the distorted image comprises:
asymmetrically cropping pixels from the flat image relative to a center of the flat image to reposition a portion of a medical instrument visible in the flat image.
